SEQUENCE LISTING (21 sequences)

SEQ ID NO: 1 - DNA, 2015 bp, Homo sapiens
gcgatgtcggtggccgggctgaagaagcagttccacaaagccagccagtagagatgggtttcaccatgttggccaatatggtctcgatctcctgacctcgtgatccgcctgcctcggcctcccagagtgctgggattacaggtgtgagccactgtgcccagcctcttctctcactatccgtttctgacaaatgaaaaaaatggatgtgggtcctgcagaacctgaggccagctctaaccagtctttcatggatgcatacacctgcctgcaggcgctgctcccaggttatgaggctggttcttgggtttgactctgggaatcatgctcattgatctgcttttcctaatcatgtcagtgaatacctacacgtgaagaaaactcaagctgcaccataaagctaaaggaagaaggagcttttaagtgaataagaccattttcccaatgagcagtgtcataaacattgaggactctcttaaatcagaagatgaatgaatggatggaatctttgctgggataatatgcaatcaagctaataggtgccttacctggacctcccaacagctatttagtgaaaaaataagtggtgctgaaggaactaaactagacgatgaatttcttgacatggaaaggaaaatagatgttaccaataaagttgttgcagaaattctttcaaaaaccactgaatatcttcagccaaatccagcatacagagctaagctaggaatgctgaacactgtgtcgaagatccgagggcaggtgaagaccacaggatacccgcagacggaaggcttgctgggggactgtatgctgaaatacgggaaggagctcggggaagactccacctttggcaatgcattgatagaagttggtgaatccatgaagctaatggctgaggtgaaagactctcttgatattaatgtaaagcaaacttttattgatccacttcagttactacaagataaagatttaaaagagatcgggcatcacctgaaaaagctggaaggccgccgcctggattacgattataaaaagaaacgagtaggtaagataccagacgaagaagtcagacaagcggtagaaaaatttgaagagtcaaaggagttggctgaaagaagcatgtttaactttttagaaaatgatgtagaacaagtcagccagttggctgtgttcatagaggcagcattagactatcacagacagtccacagagattctgcaggagctgcagagcaagctacagatgcgaatatcagctgcatccagtgtccccagacgagaatacaagccaaggcctgtgaaaaggagttctagtgagctcaatggagtttccaccacctctgtagtgaagacgacaggttctaacattcccatggaccagccctgctgtcgtggtctctatgactttgagccagaaaaccaaggagaattaggatttaaagaaggggacatcattacattaaccaatcaaatagatgaaaactggtatgaaggaatgatacacggagaatcgggattcttccccattaattacgtggaagtgatcgtgcctttacctcagtaaatgtgtaacacaaactctggacatactttcgtaactgaaatgaattcacaccagtgtgctctcagtgcggtgttctgtgacatcctttgctctctgaccaacttaatgacttttgtatgtgtgctctctttataatgtattttatatcactttaatttgtataaatgattttcttgtccttgctacatgaaaatattgttttcttttttgcttcctgtcctaaaagtcattggttaaatgtatttgcttcctgtggctaaaaataagtctcacccattgcagttatgtcaacgaatggcctatattcctcagctgcaatgaaatggtaacatttgaaactaagaaatgctaaatattttgtttctcgacattcctgatgacgtctggtcttttcttttcattgtattttaagcttacctgtgaatagcccaataaacatgacacactgtgttggc

SEQ ID NO: 2 - protein, 355 aa, Homo sapiens
MDGIFAGIICNQANRCLTWTSQQLFSEKISGAEGTKLDDEFLDMERKIDVTNKVVAEILSKTTEYLQPNPAYRAKLGMLNTVSKIRGQVKTTGYPQTEGLLGDCMLKYGKELGEDSTFGNALIEVGESMKLMAEVKDSLDINVKQTFIDPLQLLQDKDLKEIGHHLKKLEGRRLDYDYKKKRVGKIPDEEVRQAVEKFEESKELAERSMFNFLENDVEQVSQLAVFIEAALDYHRQSTEILQELQSKLQMRISAASSVPRREYKPRPVKRSSSELNGVSTTSVVKTTGSNIPMDQPCCRGLYDFEPENQGELGFKEGDIITLTNQIDENWYEGMIHGESGFFPINYVEVIVPLPQ

SEQ ID NO: 3 - DNA, 2349 bp, Homo sapiens
gcggcgggcggcagcatgtcggtggcggggctgaagaagcagttctacaaggcgagccagctggtcagtgagaaggtcggaggggccgaggggaccaagctggatgatgacttcaaagagatggagaagaaggtggatgtcaccagcaaggcggtgacagaagtgctggccaggaccatcgagtacctgcagcccaacccagcctcgcgggctaagctgaccatgctcaacacggtgtccaagatccggggccaggtgaagaaccccggctacccgcagtcggaggggcttctgggcgagtgcatgatccgccacgggaaggagctgggcggcgagtccaactttggtgacgcattgctggatgccggcgagtccatgaagcgcctggcagaggtgaaggactccctggacatcgaggtcaagcagaacttcattgaccccctccagaacctgtgcgagaaagacctgaaggagatccagcaccacctgaagaaactggagggccgccgcctggactttgactacaagaagaagcggcagggcaagatccccgatgaggagctacgccaggcgctggagaagttcgaggagtccaaggaggtggcagaaaccagcatgcacaacctcctggagactgacatcgagcaggtgagtcagctctcggccctggtggatgcacagctggactaccaccggcaggccgtgcagatcctggacgagctggcggagaagctcaagcgcaggatgcgggaagcttcctcacgccctaagcgggagtataagcccaagccccgggagccctttgaccttggagagcctgagcagtccaacgggggcttcccctgcaccacagcccccaagatcgcagcttcatcgtctttccgatcttccgacaagcccatccggacccctagccggagcatgccgcccctggaccagccgagctgcaaggcgctgtacgacttcgagcccgagaacgacggggagctgggcttccatgagggcgacgtcatcacgctgaccaaccagatcgatgagaactggtacgagggcatgctggacggccagtcgggcttcttcccgctcagctacgtggaggtgcttgtgcccctgccgcagtgactcacccgtgtccccgccccgcccctccgtccacactggccggcaccccctgctgggtctcctgcattccacggagcccctgctgccagggcggtgtctgagcctgccggcgccacctgggccccggcccttgaggtactccctgagcaggaccccacacttgggtgggggggcttatctgggtgggtggggatgcctgtttacactagcgctgactcccaacggtgacggctcccttccccactccatggcgccagcctcctcccccgctccccaacttctcgcccagctggccgaggcggggcaacactaaggtgctcttagaaacactaatgttcctctggggcagcccccacctccgtcctgacccgacgggggcccggcccactgcctaccctcgagtcccgcagccttaacaggatgggatcgagggtccccatggggtggctcagagataggaccctggttttaaatccctcccagcctggtgctggtgatgggccctggccctactccagggccaatgcacccccgcctcacacacgcactccttctcctcaaggccagggcagagggcctcaccgcctcccgggcctgctgtcagcttgcagcccggggacagaggccagctgggatctgcctgaggacagagaacatggtctcctgcagggccctgcctcccaagccccgccctcagaaagccaagtaccttttcagctttttaactgcccccatcccaacccagggaggcctgtgtcactctggcacaagctgccaccaccagccacccacacccaccccagcacacctcacacgggaccacagccgcgctgccgagggccaagcacaaaggttccagtgagcgcatgtcccagccctggtggccaggctccccttgctgagccgctgccacttcaccctgtgggaagtggccccagccatctcctctagaccaaggcaggcagccccgacatctgcttcctctatcgcccaatgcaaaatcgatgaaatggggagttctctgggccaggccacattcacattcccctccctctgtggtccagtgaagctccggaccccaggctctgctctgccctgccctgcacccccctcgtcagaagtacatgaggggcgcagagatgagcacacagctttgggcacggtccagggcaaactgaaatgtacgcctgaattttgtaaacagaagtattaaatgtctctttctac

SEQ ID NO: 4 - protein, 368 aa, Homo sapiens
MSVAGLKKQFYKASQLVSEKVGGAEGTKLDDDFKEMEKKVDVTSKAVTEVLARTIEYLQPNPASRAKLTMLNTVSKIRGQVKNPGYPQSEGLLGECMIRHGKELGGESNFGDALLDAGESMKRLAEVKDSLDIEVKQNFIDPLQNLCEKDLKEIQHHLKKLEGRRLDFDYKKKRQGKIPDEELRQALEKFEESKEVAETSMHNLLETDIEQVSQLSALVDAQLDYHRQAVQILDELAEKLKRRMREASSRPKREYKPKPREPFDLGEPEQSNGGFPCTTAPKIAASSSFRSSDKPIRTPSRSMPPLDQPSCKALYDFEPENDGELGFHEGDVITLTNQIDENWYEGMLDGQSGFFPLSYVEVLVPLPQ

SEQ ID NO: 5 - DNA, 2540 bp, Homo sapiens
cgccgcctccctcccgcacagcagccgccagcgcggcctcctgcaccatgtcggtggccggcctcaagaagcagttccataaagccactcagaaagtgagtgagaaggttggaggagctgaaggaaccaagctagatgatgacttcaaagagatggaaaggaaagtggatgtcaccagcagggctgtgatggaaataatgactaaaacaattgaataccttcaacccaatccagcttccagagctaagctcagcatgatcaacaccatgtcaaaaatccgtggccaggagaaggggccaggctatcctcaggcagaggcgctgctggcagaggccatgctcaaatttggaagagagcttggagatgattgcaactttggcccagcacttggtgaggtcggggaggccatgcgggaactgtcggaggtcaaagactctttggacatagaagtgaagcagaacttcattgaccctcttcagaatcttcatgacaaagatcttagggaaattcaacatcatctaaagaagttggagggtcgacgcctggattttgattataagaagaaacgacaaggcaagattccggatgaagagcttcgtcaagctctagagaaatttgatgagtctaaggaaattgctgagtcaagcatgttcaatctcttggagatggatattgaacaagtgagccagctctctgcacttgtgcaagctcagctggagtaccacaagcaggcagtccagatcctgcagcaagtcacggtcagactggaagaaagaataagacaggcttcatctcagcctagaagggaatatcaacctaaaccacgaatgagcctggagtttccaactggagacagtactcagcccaatgggggtctctcccacacaggcactcccaaaccttcaggtgtccaaatggatcagccctgctgccgagctctgtacgactttgaacctgaaaatgaaggggagttgggatttaaagagggcgatatcatcacactcactaaccaaattgatgagaactggtatgaggggatgctgcatggccattcaggcttcttccccatcaattatgtggaaattctggttgccctgccccattaggatgttatgctggctggctcgcctcctcttgacccagatagttacggttaaccactgctttggcaatgctgcttataacacatcccaagtgcaggccgcagtggtccacgtcatccagccccaccaagtgactttggttgacttgtgggctcccacaggagtcatggtgatggatgatatcctcttagcctggtgggcgtggcatgtgctttttaaaacatcatctgagaccagccagtagtcacagaactgctgtttacacagttctcaggaggctgtggtttattagaatatgaccatgagccatttcacagaaaaaccatcccaccgaagatattgtctatcaccccaggggccatctgaaggtctctttgcatttctccatgcaaagaggagaaagcttttgctttcacactgtcccttcccaaatatgtgagtcatggaattgtcaaagtaagccttccctcaccagcaaattgtctcctgatctgaatgaatttgtctcttaatgcatccatagaaaagtgttaattgtgggttcaaagcattctctgcaaataggcatctcagctcctcacacttatggctatttctgacgtatagccagttttcttccctccttgctattaaagccagagtcgtaattccaaattatttttcagtaagacagttaatcagcattattgtgagagggactgaaaagaaattctccattatgaggaattgggaagaaatctggtatccaagcttaaatttcttgctatacagaactatgtatgtatttaggctattctgaagggcacagggaaggggaacaaatatcttcacttcagttttatttgtgaattacatgtttcatgaatccatttggcacagagacacaaggaagaaaacactagtaaccaactttccactagttcatatactgagaaacagtaaatacctttcctttccacttttaccctgtgttctttgaacatcatttgtgcagattctgccctcaatgaggaccaaataaagatgatttttgtgcttagcagtttaaggtatatggctggcatatgcaaaactctttcccaattcagtcgctacttttacttctgccctttctatccatcgtcttcattttgtgtgtacagtgctgtgtgtaagcttatcagtgtgtttttttatttgtatcagtcatgaaagtcctgttaggtatccagagttctatttatctagctgtacagactctttcagaggtttaacgtgctgcttccgatgtgccacctgcagtagtggatcatgtggagtgaaaggcaaatcttactgcttaatgtataaactctcaccacaggaagcatcgctgtttccaataaatattgctgaagacagaaaaaaaaaaaaaaaaaaaaaaaaaaaaaaaaaaaaaaaaaaaaaaaaaaaaaaaaa

SEQ ID NO: 6 - protein, 352 aa, Homo sapiens
MSVAGLKKQFHKATQKVSEKVGGAEGTKLDDDFKEMERKVDVTSRAVMEIMTKTIEYLQPNPASRAKLSMINTMSKIRGQEKGPGYPQAEALLAEAMLKFGRELGDDCNFGPALGEVGEAMRELSEVKDSLDIEVKQNFIDPLQNLHDKDLREIQHHLKKLEGRRLDFDYKKKRQGKIPDEELRQALEKFDESKEIAESSMFNLLEMDIEQVSQLSALVQAQLEYHKQAVQILQQVTVRLEERIRQASSQPRREYQPKPRMSLEFPTGDSTQPNGGLSHTGTPKPSGVQMDQPCCRALYDFEPENEGELGFKEGDIITLTNQIDENWYEGMLHGHSGFFPINYVEILVALPH

SEQ ID NO: 7 - DNA, 20 bp, Mus musculus
aacgagtaaattgcgcccat

SEQ ID NO: 8 - DNA, 19 bp, Mus musculus
caaagggtgcgttcccact

SEQ ID NO: 9 - DNA, 26 bp, Mus musculus
  misc_feature (1)..(1): FAM reporter dye
cgaatggcctgggtagtccttgactg

SEQ ID NO: 10 - DNA, 21 bp, Mus musculus
tgctttggtaatgctgcttcc

SEQ ID NO: 11 - DNA, 20 bp, Mus musculus
gtgggcttggtgactcatcc

SEQ ID NO: 12 - DNA, 22 bp, Mus musculus
  misc_feature (1)..(1): FAM reporter dye
acatcacgaatgcaggccgcag

SEQ ID NO: 13 - DNA, 21 bp, Mus musculus
cgacgagaactggtatgaggg

SEQ ID NO: 14 - DNA, 21 bp, Mus musculus
gcacgtagctgagtgggaaga

SEQ ID NO: 15 - DNA, 21 bp, Drosophila melanogaster
  misc_feature (1)..(1): FAM reporter dye
atgctgcacggccaatcaggc

SEQ ID NO: 16 - DNA, 21 bp, Mus musculus
ggcgccgccgcgatgtcggtg

SEQ ID NO: 17 - DNA, 27 bp, Mus musculus
taggtccaaaagacacatttacggagg

SEQ ID NO: 18 - DNA, 18 bp, Artificial Sequence
  oligonucleotide primer with homology to plasmid containing mouse transgene
tgccagggagaaccaaag

SEQ ID NO: 19 - DNA, 21 bp, Artificial Sequence
  oligonucleotide primer with homology to plasmid containing mouse transgene
gcctcaccaacatctaccaac

SEQ ID NO: 20 - protein, 347 aa, Homo sapiens
MSVAGLKKQFHKASQLFSEKISGAEGTKLDDEFLDMERKIDVTNKVVAEILSKTTEYLQPNPAYRAKLGMLNTVSKIRGQVKTTGYPQTEGLLGDCMLKYGKELGEDSTFGNALIEVGESMKLMAEVKDSLDINVKQTFIDPLQLLQDKDLKEIGHHLKKLEGRRLDYDYKKKRVGKIPDEEVRQAVEKFEESKELAERSMFNFLENDVEQVSQLAVFIEAALDYHRQSTEILQELQSKLQMRISAASSVPRREYKPRPVKRSSSELNGVSTTSVVKTTGSNIPMDQPCCRGLYDFEPENQGELGFKEGDIITLTNQIDENWYEGMIHGESGFFPINYVEVIVPLPQ

SEQ ID NO: 21 - protein, 368 aa, Drosophila melanogaster
MAFAGLKKQINKANQYMTEKMGGAEGTKLDMDFMEMERKTDVTVELVEELQLKTKEFLQPNPTARAKMAAVKGISKLSGQAKSNTYPQPEGLLAECMLTYGKKLGEDNSVFAQALVEFGEALKQMADVKYSLDDNIKQNFLEPLHHMQTKDLKEVMHHRKKLQGRRLDFDCKRRRQAKDDEIRGAEDKFGESLQLAQVGMFNLLENDTEHVSQLVTFAEALYDFHSQCADVLRGLQETLQEKRSEAESRPRNEFVPKTLLDLNLDGGGGGLNEDGTPSHISSSASPLPSPMRSPAKSMAVTPQRQQQPCCQALYDFEPENPGELAFKENDIITLLNRVDDNWFEGAVNGRTGYFPQSRVSFVFPPEFF

(19) United States
(12) Patent Application Publication
(10) Pub. No.: US 2005/0176659 A1
(43) Pub. Date: Aug. 11, 2005

(54) Endophilin homologous proteins involved in the regulation of energy homeostasis

(76) Inventors: Bettina Rudolph, Hannover (DE); Ursula Hoffmann, Juhnde (DE); Karsten Eulenberg, Vom-Stein (DE); Roland Wehr, Gottingen (DE); Gunter Bronner, Gottingen (DE); Arnd Steuernagel, Gottingen (DE); Thomas Hader, Gottingen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/494,931

(22) PCT Filed: Nov. 8, 2002
(86) PCT No.: PCT/EP02/12517

(30) Foreign Application Priority Data
Nov. 8, 2001 (EP) 011 26 682.2

(51) Int. Cl. A61K 48/00; C12Q 1/68

(52) U.S. Cl. 514/44; 435/6

(57) ABSTRACT

The present invention discloses Endophilin homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of diseases and disorders, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea.

This invention relates to the use of nucleic sequences encoding Endophilin proteins, and the polypeptides encoded thereby, and to the use thereof or effectors of Endophilin in the diagnosis, study, prevention, and treatment of diseases and disorders related to body-weight regulation, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. Especially preferred is the use of Endophilin nucleic acid sequences and polypeptides or effectors of Endophilin in the modulation of adipogenesis.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly understood human disease that becomes a major health problem more and more relevant for western society. Obesity is defined as body weight more than 20% in excess of the ideal body weight, frequently resulting in a significant impairment of health. It is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors. As such, it is a complex disorder that must be addressed on several fronts to achieve lasting positive clinical outcome. Since obesity is not to be considered as a single disorder but as a heterogeneous group of conditions with (potential) multiple causes, it is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

The molecular factors regulating food intake and body weight balance are incompletely understood. Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity (Friedman and Leibel, 1990, Cell 69: 217-220). In the ob mouse a single gene mutation (obese) results in profound obesity, which is accompanied by diabetes (Friedman et. al., 1991, Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses for a specific gene involved in the regulation of body-weight, energy homeostasis, metabolism, and obesity, and thus in disorders related thereto such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. The present invention describes human endophilin genes as being involved in those conditions mentioned above.

In this invention we particularly refer to Endophilin as human Endophilin 3, Endophilin 1, or Endophilin 2 or mouse Endophilin 3, Endophilin 1, or Endophilin 2 or Drosophila Endophilin A or Endophilin B. or variants thereof. This includes Drosophila and mammalian, preferably human, homolog polypeptides or proteins or sequences encoding those proteins. Polynucleotides encoding a protein with homologies to Endophilin are suitable to investigate diseases and disorders as described above. Further, new compositions useful in diagnosis, treatment, and prognosis of diseases and disorders as described above are provided.

The endophilin (EEN; SH3p4/p8/p13) family of proteins have been described to function in intracellular signalling, more specifically, in clathrin-mediated endocytosis. Studies on the endocytosis of synaptic vesicles have shown the essential role of endophilin in vesicle formation (see, Ringstad et al. Neuron 1999 24(1):143-54). Endophilins are SH3 domain-containing cytosolic proteins acting as acyltransferases. For example, Endophilin 1 is converting lysophosphatidic acid into phosphatidic acids by addition of the fatty acid arachidonate (see, for example, review by Huttner & Schmidt Curr Opin Neurobiol 2000 10(5):543-51). Endophilin family members are highly expressed in brain, concentrated in nerve terminals, and found in complexes with protein implicated in synaptic vesicle endocytosis, the polyphosphoinositide phosphatase synaptojanin and the GTPase dynamin (see, for example, Ringstad et al. J Biol Chem 2001 2;276(44):40424-30). In addition to the role of the endophilin protein family in synaptic vesicle formation, a function of endophilins in beta 1-adrenergic receptor signaling has been shown (see, for example, Tang et al., Proc Natl Acad Sci USA 1999 26;96(22):12559-64).

So far, it has not been described that Endophilin (which particularly refers to human Endophilin 3, Endophilin 1, or Endophilin 2, or variants thereof) is involved in the regulation of energy homeostasis and body-weight regulation and related disorders, and thus, no functions in metabolic diseases and other diseases as listed aboved have been discussed.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

The present invention discloses that Endophilin homologous proteins are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences in the diagnosis, study, prevention, and treatment of diseases and disorders, for example, but not limited to, metabolic diseases such as obesity and diabetes as well as related disorders.

The term ‘GenBank Accession number’ relates to NCBI GenBank database entries (Benson et al, Nucleic Acids Res. 28, 2000, 15-18).

Endophilin homologous proteins and nucleic acid molecules coding therefore are obtainable from insect or vertebrate species, e.g. mammals or birds. Particularly preferred are human Endophilin homologous nucleic acids, particularly nucleic acids encoding a human Endophilin 3 protein, a human Endophilin 1 protein, or a human Endophilin 2 protein. Also preferred are mouse Endophilin homologous nucleic acids and polypeptides encoded thereby.

The invention particularly relates to a nucleic acid molecule encoding a polypeptide contributing to regulating the energy homeostasis and the metabolism of triglycerides, wherein said nucleic acid molecule comprises
(a) the nucleotide sequence of human Endophilin homologous nucleic acids, particularly nucleic acids encoding a human Endophilin 3 protein (Genbank Accession No. NM_{—}003027; Genbank Accession No. AF036269; EEN-B2-L1, SH3-domain GRB2-like 3); a human Endophilin 1 protein (Genbank Accession No. NM_{—}003025; Genbank Accession No. AF036268; EEN-B1, SH3-domain GRB2-like-1), or a human Endophilin 2 protein (Genbank Accession No. NM 003026; Genbank Accession No. U65999; EEN, Sh3-domain GRB2-like 2), and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of Endophilin proteins, preferably of human Endophilin proteins (GenBank Accession Numbers NP_{—}003018, NP_{—}003016, and NP_{—}003017),
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases.

The invention is based on the finding that Endophilin homologous proteins (herein referred to as Endophilin; preferably GenBank Accession Numbers NP_{—}003018, NP_{—}003016, or NP_{—}003017 for the human proteins) and the polynucleotides encoding these (for example, GenBank Accession Numbers NM_{—}003027, NM_{—}003025, or NM_{—}003026 for the human cDNAs), are involved in the regulation of triglyceride storage and therefore energy homeostasis. The invention describes the use of these compositions comprising the nucleic acids, polypeptides and effectors thereof, e.g. antibodies, aptamers, anti-sense molecules, ribozymes, RNAi molecules, peptides, low-molecular weight organic-molecules and other receptors recognizing the nucleic acid or the polypeptide for the diagnosis, study, prevention, or treatment of diseases and disorders related thereto, including metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

In this invention we demonstrate that the correct gene dose of Endophilin is essential for maintenance of energy homeostasis. The fly Drosophila melanogaster was used as model organism for the identification of proteins involved in the energy homeostasis. Drosophila melanogaster is one of the most intensively studied organisms in biology and serves as a model system for the investigation of many developmental and cellular processes common to higher eukaryotes, including humans (see, for example, Adams et al., Science 287: 2185-2195 (2000)). The success of Drosophila melanogaster as a model organism is largely due to the power of forward genetic screens to identify the genes that are involved in a biological process (see, Johnston Nat Rev Genet 3: 176-188 (2002); Rorth, Proc Natl Acad Sci USA 93: 12418-12422 (1996)).

One resource for screening was a publicly available as well as a proprietary Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells. Obese people mainly show a significant increase in the content of triglycerides. In order to isolate genes with a function in energy homeostasis, several thousand EP-lines were tested for their triglyceride content after a prolonged feeding period (see Examples for more detail). Lines with significantly changed triglyceride content were selected as positive candidates for further analysis.

Male flies heterozygous for the integration of vectors for Drosophila lines EP(3)0593 integration were analyzed in an assay measuring the triglyceride contents of these flies, illustrated in more detail in the EXAMPLES. The result of the triglyceride content analysis is shown in FIG. 1. The average triglyceride level of the proprietary fly collection in which the EP(3)0593 lines was found is shown as 100% in FIG. 1 (First column, EP-control males). The average increase of triglyceride content of the heterozygous lethal Drosophila line EP(3)0593(referred to as ‘EP(3)0593’ in this invention) is 100% (see FIG. 1, second column, ‘EP(3)0593/TM3,Sb males’). It was found in this invention that heterozygous EP(3)0593 flies have a significant higher triglyceride content than the control flies tested. The increase of triglyceride content due to the potential loss of a gene function suggests potential gene activities in energy homeostasis in a dose dependent manner that controls the amount of energy stored as triglycerides.

Nucleic acids encoding the Endophilin protein of the present invention were identified using a plasmid-rescue technique. Genomic DNA sequences were isolated that are localised to the EP vector (herein EP(3)0593) integration. Using those isolated genomic sequences public databases like Berkeley Drosophila Genome Project (GadFly) were screened thereby confirming the heterozygous lethal integration side of the EP(3)0593 vector 45 base pairs 5′ of the cDNA of the Endophilin A gene, identified as Berkeley Drosophila Genome Project Accession Nr. CG1 4296 (FIG. 2). FIG. 2 shows the molecular organisation of this gene locus. The chromosomal localization site of the integration of the vector of EP(3)0593 is at gene locus 3R, 91 D4. In FIG. 2, genomic DNA sequence is represented as a black dotted line in the middle that includes the integration site of EP(3)0593. Numbers represent the coordinates of the genomic DNA (starting at position 14653366 on chromosome 3R, ending at position 14678366 on chromosome 3R). Grey bars on the two “cDNA”-lines represent the predicted genes (as predicted by the Berkeley Drosophila Genome Project, GadFly and by Magpie). Predicted exons of the Drosophila cDNA (Berkeley Drosophila Genome Project Accession Nr. CG14296) are shown as dark grey bars and predicted introns as light grey bars. The boxes referring to expressed sequence tag (EST) clones ‘DGC GH12907’ (dark grey bars) and to EST clones ‘Clot 5557_{—}3 & 1’ (light grey bars) show that endophilin is a transcribed gene.

Expression profiling studies (see Examples for more detail) confirm the particular relevance of Endophilin 3 and Endophilin 1 as regulators of energy metabolism in mammals. In comparison to Endophilin 2, which is rather ubiquitously expressed, both Endophilin 3 and Endophilin 1 transcripts are more restricted in neuronal tissues of mammals. However, both Endophilin 3 and Endophilin 1 are also clearly expressed in white adipose tissue (WAT) and brown adipose tissue (BAT) (see FIG. 4A and FIG. 5A, respectively), indicating a role in the regulation of energy homeostasis.

Further, we show that mammalian homologues of the Drosophila Endophilin gene, particularly Endophilin 3 and. Endophilin 1, are regulated by metabolic conditions such as fasting and genetically induced obesity. For example, the expression of Endophilin 3 is strongly upregulated in liver of fasted mice (see FIG. 4B). In addition, a marked downregulation can be observed in the metabolically active tissue (for example, brown adipose tissue (BAT)) of genetically obese (ob/ob) as well as of fasted mice (see FIG. 4B). Endophilin 1 is strongly downregulated in white adipose tissue (WAT) of genetically obese db/db mice (see FIG. 5B).

The most prominent response with regard to metabolically active tissues can be seen in mice kept under a high fat diet. In those mice, the expression of Endophilin 3 as well as Endophilin 1 is significantly reduced in white adipose tissue (see FIG. 4C and FIG. 5C, respectively), supporting the finding that Endophilin 3 and/or Endophilin 1 is a modulator (for example, inhibitor) of adipogenesis.

In addition, we show in this invention that the Endophilin 3 mRNA is strongly down-regulated during adipocyte differentiation in vitro (see EXAMPLES for more detail), demonstrating a role as modulator (for example, inhibitor) of adipocyte lipid accumulation. With regard to changes in expression intensity during the differentiation of preadipocytes to adipocytes, a strong reduction in relative signal intensity can be observed for Endophilin 3 during the in vitro differentiation program of 3T3-L1 as well as 3T3-F442A cells (see FIG. 4D and FIG. 4E).

Thus, we conclude that Endophilin 3 (or variants thereof) or Endophilin 1 (or variants thereof) has a function in the metabolism of mature adipocytes.

The present invention is further describing a polypeptide comprising the amino acid sequence of Endophilin. A comparison (Clustal X 1.8 analysis) between the Endophilin proteins of different species (human and Drosophila) was conducted (See FIG. 3). The sequence similarities between Drosophila Endophilin A (GadFly Accession Number CG14296) and human homologs (for Endophilin 3, NP_{—}003018; for Endophilin 1, NP_{—}003016; for Endophilin 2, NP_{—}003017) are as described below:
CG14296 vs. GenBank Accession No. NP_{—}003018 50% identity, 66% similarity (366 amino acids)
CG14296 vs. GenBank Accession No. NP_{—}003016 49% identity, 67% similarity (370 amino acids)
CG14296 vs. GenBank Accession No. NP_{—}003017 48% identity, 65% similarity (369 amino acids)

Based upon homology, an Endophilin protein of the invention and each homologous protein or peptide may share at least some activity.

No functional data for such proteins relating to the regulation of body weight control and related metabolic diseases such as obesity are available in the prior art. Our Drosophila data as well as results from the expression profiling in different mouse models clearly show that Endophilin is involved in the regulation of the metabolism in fly and mammals. In addition, as shown in FIG. 7C, the basal glucose uptake of cells over-expressing endophillin 3 is significantly increased during adipogenesis. This increase in glucose and therefore energy uptake of the cells is most likely the reason for the increased glycogen and triglyceride levels during adipocyte differentiation (see FIGS. 7A and 7B, respectively). Endophillin 3 does not seem to influence insulin stimulated glucose uptake but clearly has an effect on the glucose uptake of adipocytes, confirming a role in diabetes and related metabolic disorders.

The invention also encompasses polynucleotides that encode Endophilin and homologous proteins. Accordingly, any nucleic acid sequence, which encodes the amino acid sequences of Endophilin, can be used to generate recombinant molecules that express Endophilin. In a particular embodiment, the invention encompasses human Endophilin homologous nucleic acids, particularly nucleic acids encoding a human Endophilin 3 protein, a human Endophilin 1 protein, or a human Endophilin 2 protein. Also preferred are mouse Endophilin homologous nucleic acids and polypeptides encoded thereby. It will be appreciated by those skilled in the art that as a result of the degeneracy of the genetic code, a multitude of nucleotide sequences encoding Endophilin, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequences of naturally occurring Endophilin, and all such variations are to be considered as being specifically disclosed. Although nucleotide sequences which encode Endophilin and its variants are preferably capable of hybridising to the nucleotide sequences of the naturally occurring Endophilin under appropriately selected conditions of stringency, it may be advantageous to produce nucleotide sequences encoding Endophilin or its derivatives possessing a substantially different codon usage. Codons may be selected to increase the rate at which expression of the peptide occurs in a particular prokaryotic or eukaryotic host in accordance with the frequency with which particular codons are utilised by the host. Other reasons for substantially altering the nucleotide sequence encoding Endophilin and its derivatives without altering the encoded amino acid sequences include the production of RNA transcripts having more desirable properties, such as a greater half-life, than transcripts produced from the naturally occurring sequences. The invention also encompasses production of DNA sequences, or portions thereof, which encode Endophilin and its derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding Endophilin any portion thereof.

Also encompassed by the invention are polynucleotide sequences that are capable of hybridising to the claimed nucleotide sequences, and in particular, human Endophilin homologous nucleic acids, particularly nucleic acids encoding a human Endophilin 3 protein, a human Endophilin 1 protein, or a human Endophilin 2 protein under various conditions of stringency. Hybridisation conditions are based on the melting temperature (Tm) of the nucleic acid binding complex or probe, as taught in Wahl, G. M. and S. L. Berger (1987: Methods Enzymol. 152:399-407) and Kimmel, A. R. (1987, Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding Endophilin which are encompassed by the invention include deletions, insertions, or substitutions of different nucleotides resulting in a polynucleotide that encodes the same or a functionally equivalent Endophilin.

The encoded proteins may also contain deletions, insertions, or substitutions of amino acid residues, which produce a silent change and result in a functionally equivalent Endophilin. Deliberate amino acid substitutions may be made on the basis of similarity in polarity, charge, solubility, hydrophobicity, hydrophilicity, and/or the amphipathic nature of the residues as long as the biological activity of Endophilin is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine. Furthermore, the invention relates to peptide fragments of the proteins or derivatives thereof such as cyclic peptides, retro-inverso peptides or peptide mimetics having a length of at least 4, preferably 6 and up to 50 amino acids.

Also included within the scope of the present invention are alleles of the genes encoding Endophilin. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

Methods for DNA sequencing which are well known and generally available in the art may be used to practice any embodiments of the invention. The methods may employ such enzymes as the Klenow fragment of DNA polymerase 1, SEQUENASE DNA Polymerase (US Biochemical Corp, Cleveland Ohio), Taq polymerase (Perkin Elmer), thermostable T7 polymerase (Amersham, Chicago, Ill.), or combinations of recombinant polymerases and proof-reading exonucleases such as the ELONGASE Amplification System (GIBCO/BRL, Gaithersburg, Md.). Preferably, the process is automated with machines such as the Hamilton MICROLAB 2200 (Hamilton, Reno Nev.), Peltier thermal cycler (PTC200; MJ Research, Watertown, Mass.) and the ABI 377 DNA sequencers (Perkin Elmer).

The nucleic acid sequences encoding Endophilin may be extended utilising a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences, which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions. Capillary electrophoresis systems, which are commercially available, may be used to analyse the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. Output/light intensity may be converted to electrical signal using appropriate software (e.g. GENOTYPER and SEQUENCE NAVIGATOR, Perkin Elmer) and the entire process from loading of samples to computer analysis and electronic data display may be computer controlled. Capillary electrophoresis is especially preferable for the sequencing of small pieces of DNA, which might be present in limited amounts in a particular sample.

In another embodiment of the invention, polynucleotide sequences or fragments thereof which encode Endophilin, or fusion proteins or functional equivalents thereof, may be used in recombinant DNA molecules to direct expression of Endophilin in appropriate host cells. Due to the inherent degeneracy of the genetic code, other DNA sequences, which encode substantially the same, or a functionally equivalent amino acid sequence may be produced and these sequences may be used to clone and express Endophilin. As will be understood by those of skill in the art, it may be advantageous to produce Endophilin-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. The nucleotide sequences of the present invention can be engineered using methods generally known in the art in order to alter Endophilin encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences encoding Endophilin may be ligated to a heterologous sequence to encode a fusion protein. For example, to screen peptide libraries for inhibitors of Endophilin activities, it may be useful to produce chimeric Endophilin proteins that can be recognised by a commercially available antibodies. A fusion protein may also be engineered to contain a cleavage site located between the Endophilin encoding sequence and the heterologous protein sequences, so that Endophilin may be cleaved and purified away from the heterologous moiety. In another embodiment, sequences encoding Endophilin may be synthesised, in whole or in part, using chemical methods well known in the art (see Caruthers, M. H. et al. (1980) Nucl. Acids Res. Symp. Ser. 7:215-223, Horn, T. et al. (1980) Nucl. Acids Res. Symp. Ser. 7:225-232). Alternatively, the proteins themselves may be produced using chemical methods to synthesise the amino acid sequence of Endophilin, or a portion thereof. For example, peptide synthesis can be performed using various solid-phase techniques (Roberge, J. Y. et al. (1995) Science 269:202-204) and automated synthesis may be achieved, for example, using the ABI 431A peptide synthesiser (Perkin Elmer). The newly synthesised peptide may be substantially purified by preparative high performance liquid chromatography (e.g., Creighton, T. (1983) Proteins, Structures and Molecular Principles, W H Freeman and Co., New York, N.Y.). The composition of the synthetic peptides may be confirmed by amino acid analysis or sequencing (e.g., the Edman degradation procedure; Creighton, supra). Additionally, the amino acid sequences of Endophilin, or any part thereof, may be altered during direct synthesis and/or combined using chemical methods with sequences from other proteins, or any part thereof, to produce a variant polypeptide.

In order to express a biologically active Endophilin, the nucleotide sequences encoding Endophilin functional equivalents, may be inserted into appropriate expression vectors, i.e., a vector, which contains the necessary elements for the transcription and translation of the inserted coding sequence. Methods, which are well known to those skilled in the art, may be used to construct expression vectors containing sequences encoding Endophilin and appropriate transcriptional and translational control elements. These methods include in vitro recombinant DNA techniques synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in. Molecular Biology, John Wiley & Sons, New York, N.Y.

A variety of expression vector/host systems may be utilised to contain and express sequences encoding Endophilin. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems. The “control elements” or “regulatory sequences” are those non-translated regions of the vector-enhancers, promoters, 5′ and 3′ untranslated regions which interact with host cellular proteins to carry out transcription and translation. Such elements may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters and enhancers derived from the genomes of plant cells (e.g., heat shock, RUBISCO; and storage protein genes) or from plant viruses (e.g., viral promoters and leader sequences) may be cloned into the vector. In mammalian cell systems, promoters from mammalian genes or from mammalian viruses are preferable. If it is necessary to generate a cell line that contains multiple copies of the sequences encoding Endophilin, vectors based on SV40 or EBV may be used with an appropriate selectable marker.

In bacterial systems, a number of expression vectors may be selected depending upon the use intended for Endophilin. For example, when large quantities of Endophilin are needed for the induction of antibodies, vectors, which direct high level expression of fusion proteins that are readily purified, may be used. Such vectors include, but are not limited to, the multifunctional E. coli cloning and expression vectors such as the BLUESCRIPT phagemid (Stratagene), in which the sequence encoding Endophilin may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. Vectors of the pGEX series (Amersham Bioscience, Uppsala, Sweden) may also be used to express foreign polypeptides as fusion proteins with Glutathione S-Transferase (GST). In general, such fusion proteins are soluble and can easily be purified from lysed cells by adsorption to glutathione-agarose beads followed by elution in the presence of free glutathione. In another expression system, the coding sequence of endophilin may be expressed in frame with six or more consequtive histidin residues, which may be located close to the amino terminus, carboxy terminal or internally. A variety of vectors, which include, but are not limited to, the pQE series (Qiagen, Hilden, Germany), pBAD and pTrc series (Invitrogen, Carlsbad, Calif.) and pET series (Novagen, Madison, Wis.) may be suitable for expression of endophilin in E. coli and a subsequent purification from the lysed cells by imobilized metal ion affinity chromatography (IMAC).

Proteins made in such systems may be designed to include heparin, thrombin, or factor XA protease cleavage sites so that the cloned polypeptide of interest can be released from the GST moiety at will. In the yeast, Saccharomyces cerevisiae, a number of vectors containing constitutive or inducible promoters such as alpha factor, alcohol oxidase, and PGH may be used. For reviews, see Ausubel et al., (supra) and Grant et al. (1987) Methods Enzymol. 153:516-544.

In cases where plant expression vectors are used, the expression of sequences encoding Endophilin may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express Endophilin. For example, in one such system, Autographa californica nuclear polyhedrosis virus (AcNPV) is used as a vector to express foreign genes in Spodoptera frugiperda cells or in Trichoplusia larvae. The sequences encoding Endophilin may be cloned into a non-essential region of the virus, such as the polyhedrin gene, and place under control of the polyhedrin promoter. Successful insertions of Endophilin will render the polyhedrin gene inactive and produce recombinant virus lacking coat protein. The recombinant viruses may then be used to infect, for example, S. frugiperda cells of Trichoplusia larvae in which Endophilin may be expressed (Engelhard, E. K. et al. (1994) Proc. Nat. Acad. Sci. 91:3224-3227).

In mammalian host cells, a number of non-viral-based or viral-based expression systems may be utilised. In cases where an adenovirus is used as an expression vector, sequences encoding Endophilin may be ligated into an adenovirus transcription/translation complex consisting of the late promoter and tripartite leader sequence. Insertion in a non-essential El or E3 region of the viral genome may be used to obtain viable viruses which are capable of expressing Endophilin in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding Endophilin. Such signals include the ATG initiation codon and adjacent sequences. In cases where sequences encoding Endophilin, its initiation codons, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express Endophilin may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells, which successfully express the introduced sequences. Resistant clones of stably transformed cells may be proliferated using tissue culture techniques appropriate to the cell type. Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:81 7-23) genes, which can be employed in tk-or aprt-,cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilise indole in place of tryptophan, or hisD, which allows cells to utilise histinol in place of histidine (Hartman, S. C. and R. C. Mulligan (1 988) Proc. Natl. Acad. Sci. 85:8047-51). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequences encoding Endophilin are inserted within a marker gene sequence, recombinant cells containing sequences encoding Endophilin can be identified by the absence of marker gene function. Alternatively, a marker gene can be placed in tandem with sequences encoding Endophilin under the control of a single promoter. Expression of the marker gene in response to induction or selection usually indicates expression of the tandem gene as well. Alternatively, host cells, which contain the nucleic acid sequences encoding Endophilin and express Endophilin, may be identified by a variety of procedures known to those of skill in the art. These procedures include, but are not limited to, DNA-DNA, or DNA-RNA hybridisation and protein bioassay or immunoassay techniques which include membrane, solution, or chip based technologies for the detection and/or quantification of nucleic acid or protein.

The presence of polynucleotide sequences encoding Endophilin can be detected by DNA-DNA or DNA-RNA hybridisation and/or amplification using probes or portions or fragments of polynucleotides encoding Endophilin. Nucleic acid amplification based assays involve the use of oligonucleotides or oligomers based on the sequences encoding Endophilin to detect transformants containing DNA or RNA encoding Endophilin. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of Endophilin, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilising monoclonal antibodies reactive to two non-interfering epitopes on Endophilin is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983;, J. Exp. Med. 158:1211-1216).

A wide variety of labels and conjugation techniques are known by those skilled in the art and may be used in various nucleic acid and amino acid assays. Means for producing labelled hybridisation or PCR probes for detecting sequences related to polynucleotides encoding Endophilin include oligo-labelling, nick translation, end-labelling or PCR amplification using a labelled nucleotide.

Alternatively, the sequences encoding Endophilin, or any portions thereof may be cloned into a vector for the production of an mRNA probe. Such vectors are known in the art, are commercially available, and may be used to synthesise RNA probes in vitro by addition of an appropriate RNA polymerase such as T7, T3, or SP6 and labelled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding Endophilin may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode Endophilin may be designed to contain signal sequences, which direct secretion of Endophilin through a prokaryotic or eukaryotic cell membrane. Other recombinant constructions may be used to join sequences encoding Endophilin to nucleotide sequence encoding a polypeptide domain, which will facilitate purification of soluble proteins. Such purification facilitating domains include, but are not limited to, metal chelating peptides such as histidine-tryptophan modules that allow purification on immobilised metals, protein A domains that allow purification on immobilised immunoglobulin, and the domain utilised in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and Endophilin may be used to facilitate purification. One such expression vector provides for expression of a fusion protein containing Endophilin and a nucleic acid encoding 6 histidine residues preceding a Thioredoxine or an Enterokinase cleavage site. The histidine residues facilitate purification on IMIAC (immobilised metal ion affinity chromatography as described in Porath, J. et al. (1992, Prot. Exp. Purif. 3: 263-281)) while the Enterokinase cleavage site provides a means for purifying Endophilin from the fusion protein. A discussion of vectors which contain fusion proteins is provided in Kroll, D. J. et al. (1993; DNA Cell Biol. 12:441-453). In addition to recombinant production, fragments of Endophilin may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesiser (Perkin Elmer). Various fragments of Endophilin may be chemically synthesised separately and combined using chemical methods to produce the full length molecule.

Diagnostics and Therapeutics

The data disclosed in this invention show that the nucleic acids and proteins of the invention and effector molecules thereof are useful in diagnostic and therapeutic applications implicated, for example but not limited to, in metabolic disorders such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. Hence, diagnostic and therapeutic uses for the Endophilin nucleic acids and proteins of the invention are, for example but not limited to, the following: (i) protein therapeutic, (ii) small molecule drug target, (iii) antibodytarget (therapeutic, diagnostic, drug targeting/cytotoxic antibody), (iv) diagnostic and/or prognostic marker,. (v) gene therapy (gene delivery/gene ablation), (vi) research tools, and (vii) tissue regeneration in vitro and in vivo (regeneration for all these tissues and cell types composing these tissues and cell types derived from these tissues). A particularly preferred application is the modulation, e.g. inhibition of adipogenesis.

The nucleic acids and proteins of the invention and effectors thereof are useful in diagnostic and therapeutic applications implicated in various applications as described below. For example, but not limited to, cDNAs encoding the Endophilin proteins of the invention and particularly their human homologues may be useful in gene therapy, and the Endophilin proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acid(s) encoding the Endophilin protein(s) of the invention, or fragments thereof, may further be useful in diagnostic applications, wherein the presence or amount of the nucleic acids or the proteins are to be assessed. These materials are further useful in the generation of antibodies that bind immunospecifically to the substances of the invention for use in therapeutic or diagnostic methods.

For example, in one aspect, antibodies which are specific for Endophilin may be used directly as an antagonist, or indirectly as a targeting or delivery mechanism for bringing a pharmaceutical agent to cells or tissue which express Endophilin. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunised by injection with Endophilin any fragment or oligopeptide thereof which has immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to Endophilin have an amino acid sequence consisting of at least five amino acids, and more preferably at least 10 amino acids. It is preferable that they are identical to a portion of the amino acid sequence of the natural protein, and they may contain the entire amino acid sequence of a small, naturally occurring molecule. Short stretches of Endophilin amino acids may be fused with those of another protein such as keyhole limpet hemocyanin and antibody produced against the chimeric molecule.

Monoclonal antibodies to Endophilin may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor,. D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120). In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce Endophilin- and -specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for Endophilin, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between Endophilin and its specific antibody. A two-site, monoclonal-based immunoassay utilising monoclonal antibodies reactive to two non-interfering Endophilin epitopes is preferred, but a competitive binding assay may also be employed (Maddox, supra).

In another embodiment of the invention, the polynucleotides encoding Endophilin, or any fragment thereof, or nucleic acid effector molecules, such as aptamers, anti-sense molecules, ribozymes or RNAi molecules may be used for therapeutic purposes. In one aspect, aptamers, i.e. nucleic acid molecules, which are capable of binding to an Endophilin protein and modulating its activity, may be generated by a screening and-selection procedure involving the use of combinatorial nucleic acid libraries.

In a further aspect, antisense molecules to the polynucleotide encoding Endophilin may be used in situations in which it would be desirable to block the transcription of the mRNA. In particular, cells may be transformed with sequences complementary to polynucleotides encoding Endophilin. Thus, antisense molecules may be used to modulate Endophilin activity, or to achieve regulation of gene function. Such technology is now well know in the art, and sense or antisense oligomers or larger fragments, can be designed from various locations along the coding or control regions of sequences encoding Endophilin. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the gene encoding Endophilin. These techniques are described both in Sambrook et al. (supra) and in Ausubel et al. (supra). Genes encoding Endophilin can be turned off by transforming a cell or tissue with expression vectors which express high levels of polynucleotide or fragment thereof which encodes Endophilin. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA, or nucleic acid analogues such as PNA, to the control regions of the gene encoding Endophilin, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding Endophilin. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding Endophilin. Such DNA sequences may be incorporated into a variety of vectors with suitable RNA polymerase promoters such as T7 or SP6. Alternatively, these cDNA constructs that synthesise antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of Endophilin, antibodies to Endophilin, mimetics, agonists, antagonists, or inhibitors of Endophilin. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.). Pharmaceutical compositions can be formulated using pharmaceutically acceptable carriers well known in the art in dosages suitable for administration. Such carriers enable the pharmaceutical compositions to be formulated as tablets, pills, dragees, capsules, liquids, gels, syrups, slurries, suspensions, and the like.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilising processes. The pharmaceutical composition may be provided as a salt and can be formed with many acids, including but not limited to, hydrochloric, sulphuric, acetic, lactic, tartaric, malic, succinic, etc. After pharmaceutical compositions have been prepared, they can be placed in an appropriate container and labelled for treatment of an indicated condition. For administration of Endophilin, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example Endophilin, fragments thereof or antibodies of Endophilin, which is sufficient for treating a specific condition. Therapeutic efficacy and toxicity may be determined by standard pharmaceutical procedures in cell cultures or experimental animals, e.g. ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind Endophilin may be used for the diagnosis of conditions or diseases characterised by or associated with over- or underexpression of Endophilin, or in assays to monitor patients being treated with Endophilin, agonists, antagonists or inhibitors. The antibodies useful for diagnostic purposes may be prepared in the same manner as those described above for therapeutics. Diagnostic assays for Endophilin include methods, which utilise the antibody and a label to detect Endophilin in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring Endophilin are known in the art and provide a basis for diagnosing altered or abnormal levels of Endophilin expression. Normal or standard values for Endophilin expression are established by combining body fluids or cell extracts taken from normal mammalian subjects, preferably human, with antibody to Endophilin under conditions suitable for complex formation. The amount of standard complex formation may be quantified by various methods, but preferably by photometric means. Quantities of Endophilin expressed in control and disease samples e.g. from biopsied tissues are compared with the standard values. Deviation between standard and subject values establishes the parameters for diagnosing disease.

In another embodiment of the invention, the polynucleotides encoding Endophilin may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of Endophilin may be correlated with disease. The diagnostic assay may be used to distinguish between absence, presence, and excess expression of Endophilin, and to monitor regulation of Endophilin levels during therapeutic intervention.

In one aspect, hybridisation with PCR probes which are capable of detecting polynucleotide sequences, including genomic sequences, encoding Endophilin closely related molecules, may be used to identify nucleic acid sequences which encode Endophilin. The specificity of the probe, whether it is made from a highly specific region, e.g., unique nucleotides in the 5′ regulatory region, or a less specific region, e.g., especially in the 3′ coding region, and the stringency of the hybridisation or amplification (maximal, high, intermediate, or low) will determine whether the probe identifies only naturally occurring sequences encoding Endophilin, alleles, or related sequences. Probes may also be used for the detection of related sequences, and should preferably contain at least 50% of the nucleotides from any of the Endophilin encoding sequences. The hybridisation probes of the subject invention may be DNA or RNA and are preferably derived from the nucleotide sequence of human Endophilin homologous nucleic acids, particularly nucleic acids encoding a human Endophilin 3 protein, a human Endophilin 1 protein, or a human Endophilin 2 protein or from a genomic sequence including promoter, enhancer elements, and introns of a naturally occurring Endophilin gene. Means for producing specific hybridisation probes for DNAs encoding Endophilin include the cloning of nucleic acid sequences encoding Endophilin derivatives into vectors for the production of mRNA probes. Such vectors are known in the art, commercially available, and may be used to synthesise RNA probes in vitro by means of the addition of the appropriate RNA polymerases and the appropriate labelled nucleotides. Hybridisation probes may be labelled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences encoding Endophilin may be used for the diagnosis of conditions or diseases, which are associated with expression of Endophilin. Examples of such conditions or diseases include, but are not limited to, pancreatic diseases and disorders, including diabetes. Polynucleotide sequences encoding Endophilin may also be used to monitor the progress of patients receiving treatment for pancreatic diseases and disorders, including diabetes. The polynucleotide sequences encoding Endophilin may be used in Southern or Northern analysis, dot blot, or other membrane-based technologies; in PCR technologies; or in dip stick, pin, ELISA or chip assays utilising fluids or tissues from patient biopsies to detect altered Endophilin expression. Such qualitative or quantitative methods are well known in the art.

In a particular aspect, the nucleotide sequences encoding Endophilin may be useful in assays that detect activation or induction of various metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea. The nucleotide sequences encoding Endophilin may be labelled by standard methods, and added to a fluid or tissue sample from a patient under conditions suitable for the formation of hybridisation complexes. After a suitable incubation period, the sample is washed and the signal is quantitated and compared with a standard value. If the amount of signal in the biopsied or extracted sample is significantly altered from that of a comparable have hybridised with nucleotide sequences in the sample, and the presence of altered levels of nucleotide sequences encoding Endophilin in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of Endophilin, a normal or standard profile for expression is established. This may be accomplished by combining body fluids or cell extracts taken from normal subjects, either animal or human, with a sequence, or a fragment thereof, which encodes Endophilin, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders.

Additional diagnostic uses for oligonucleotides designed from the sequences encoding Endophilin may involve the use of PCR. Such oligomers may be chemically synthesised, generated enzymatically, or produced from a recombinant source. Oligomers will preferably consist of two nucleotide sequences, one with sense orientation (5′.fwdarw.3′) and another with antisense (3′.rarw.5′), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of Endophilin include radiolabelling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or colorimetric response gives rapid quantification.

In another embodiment of the invention, the nucleic acid Endophilin sequences, which encode Endophilin, may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial. chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding Endophilin on a physical chromosomal map and a specific disease, or predisposition to a specific disease, may help to delimit the region of DNA associated with that genetic disease.

The nucleotide sequences of the subject invention may be used to detect differences in gene sequences between normal, carrier or affected individuals. An analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may-also be used to detect differences in the chromosomal location due to translocation, inversion, etc. among normal, carrier or affected individuals.

In another embodiment of the invention, the proteins of the invention, its catalytic or immunogenic fragments or oligopeptides thereof, an in vitro model, a genetically altered cell or animal, can be used for screening libraries of compounds in any of a variety of drug screening techniques. One can identify effectors, e.g. receptors, enzymes, ligands or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between the proteins of the invention and the agent tested, may be measured. Agents can also be identified, which either directly or indirectly, influence the activity of the proteins of the invention. Target mechanisms could for example include the lysophosphatidic acid acyl transferase (LPAAT) activity of endophilin (see, Schmidt et al.) 1999, Nature 401: 123-124). Assays aimed at determination of LPAAT activity are well known in the art, i.e. the transfer of arachidonate from the radioactive labeled donor Arachidonoyl CoA to lysophosphatidic acid could be quantified after thin layer chromatography of the lipids by determination of the amount of reaction product, phosphatidic acid. Alternatively, monitoring of the reaction could be performed by using fluorescently labeled substrate or acceptor molecules, which can be identical to or different from Arachidonoyl CoA and lysophosphatidic acid. Determination of LPAAT activity can either be performed in cell-based assays using endophilin overexpressing cells or in in vitro assays using purified endophilin, generated as described above. Another target mechanism would be the interaction of endophilin with other cellular proteins, which could be for example, but not exclusively, the β1 adrenergic receptor (Tang et al. (1999) PNAS 96:12559-12564) or CIN85 complexed with or without Cbl (Petrelli et al. (2002) Nature 416:187-190, Soubeyran et al (2002)

Nature 41 6:183-187). Methods for determining protein-protein Interaction are well known In the art. For example binding of a fluorescently labeled peptide derived from the protein to endophilin, or vice versa, could be detected by a change In polarisation. In case that both binding partners, which can be either the full length proteins as well as one binding partner as the full length protein and the other just represented as a peptide are fluorescently labeled, bindung could be detected by fluorescence energy transfer (FRET) from one fluorophore to the other. In addition, a variety of commercially available assay principles suitable for detection of protein-protein Interaction are well known In the art, for example but not exclusively AlphaScreen (PerkinElmer) or Scintillation Proximity Assays (SPA) by Amersham. Alternatively, the Interaction of Endophilin with cellular proteins could be the basis for a cell-based screening assay, In which both proteins are fluorescently labeled and Interaction of both proteins Is detected by-analysing cotranslocation of both proteins with a cellular Imaging reader, as has been developed for example, but not exclusively, by Cellomics or EvotecOAI.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with the protein.

The nucleic acids encoding the proteins of the invention can be used to generate transgenic cell lines and animals. These transgenic animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLE 4), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a Endophilin 3, Endophilin 1, or Endophilin 2 nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

The kit may be used for diagnostic or therapeutic purposes or for screening applications as described above. The kit may further contain user instructions.

### THE FIGURES SHOW

FIG. 1 shows the increase of triglyceride content of EP(3)0593 flies caused by heterozygous lethal integration of the P-vector (in comparison to controls without integration of this vector).

FIG. 2 shows the molecular organisation of the mutated Endophilin gene locus.

FIG. 3. Endophilin Sequences

FIG. 3A. Nucleic Acid sequence of human Endophilin 3 (also referred to as SH3-domain GRB2-like 3 protein; GenBank Accession Number NM_{—}003027; SEQ ID NO. 1).

FIG. 3B. Amino Acid sequence of human Endophilin 3 (also referred to as SH3-domain GRB2-like 3 protein; GenBank Accession Number NP_{—}003018; SEQ ID NO. 2).

FIG. 3C. Nucleic Acid sequence of human Endophilin 1 (also referred to as SH3-domain GRB2-like 1 protein; GenBank Accession Number NM_{—}003025; SEQ ID NO. 3).

FIG. 3D. Amino Acid sequence of human Endophilin 1 (also referred to as SH3-domain GRB2-like 1 protein; GenBank Accession Number NP_{—}003016; SEQ ID NO. 4).

FIG. 3E. Nucleic Acid sequence of human Endophilin 2 (also referred to as SH3-domain GRB2-like 2 protein; GenBank Accession Number NM_{—}003026; SEQ ID NO. 5).

FIG. 3F. Amino Acid sequence of human Endophilin 2 (also referred to as SH3-domain GRB2-like 2 protein; GenBank Accession Number NP_{—}003017; SEQ ID NO. 6).

FIG. 3G shows the CLUSTAL X (1.81) multiple sequence alignment for Endophilin. NP_{—}003016 refers to the Accession Number for human Endophilin 1, NP_{—}003017 refers to the Accession Number for human Endophilin 2, NP_{—}003018 refers to the Accession Number for human Endophilin 3, and CG14296 refers to the GadFly Accession Number for Drosophila Endophilin A.

FIG. 4 shows the expression of Endophilin 3 in different mammalian models.

FIG. 4A. Expression of Endophilin 3 in different wildtype mouse tissues

FIG. 4B. Expression of Endophilin 3 in different mouse models: wildtype (wt), fasted mice and obese mice-(ob/ob)

FIG. 4C. Expression of Endophilin 3 in susceptible wild-type mice under a high-fat diet

FIG. 4D. Expression of Endophilin 3 during differentiation of cultured 3T3-L1 cells from pre-adipocytes to mature adipocytes

FIG. 4E. Expression of Endophilin 3 during differentiation of cultured 3T3-F422A cells from pre-adipocytes to mature adipocytes

FIG. 5 shows the expression of Endophilin 1 in different mammalian models.

FIG. 5A. Expression of Endophilin 1 in different wildtype mouse tissues

FIG. 5B. Expression of Endophilin 1 in different mouse models (in wildtype (wt) and obese (ob/ob) mice

FIG. 5C. Expression of Endophilin 1 in mice under a high-fat diet

FIG. 6. Expression of Endophilin 2 in different wildtype mouse tissues

FIG. 7 shows in vitro assays for the determination of triglyceride, glycogen and glucose levels in cells overexpressing endophilin 3.

FIG. 7A shows an increase in triglyceride levels in cells overexpressing endophilin 3 versus control cells. The Y-axis shows cellular triglyceride levels (g/mg protein) and the X-axis shows days of cell differentiation.

FIG. 7B shows an increase in glycogen levels in cells overexpressing endophilin 3. The Y-axis shows glycogen levels (shown as microg glycogen per mg protein) and the X-axis shows days of cell differentiation.

FIG. 7C shows an increase in glucose uptake in cells overexpressing endophilin 3. The Y-axis shows glucose levels (shown as disintegrations per minutes -dpm- per mg protein) and the X-axis shows days of cell differentiation.

### THE EXAMPLES ILLUSTRATE THE INVENTION

### Example 1

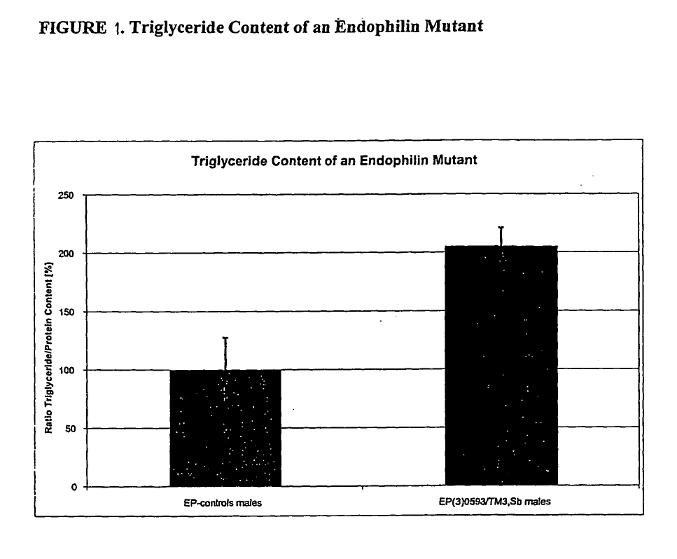

### Measurement of Triglyceride Content in Drosophila (FIG. 1)

Mutant flies are obtained from the P Insertion Mutation Stock Center, Sezged, Hungary. The flies are grown under standard conditions known to those skilled in the art. In the course of the experiment, additional feedings with bakers yeast (Saccharomyces cerevisiae) are provided. The average increase of triglyceride content of Drosophila containing the EP(3)0593 vectors in heterozygous integration was investigated in comparison to control flies (FIG. 1). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assay was repeated several times. The average triglyceride level of all flies of the EP collection (referred to as ‘EP-controls males’) is shown as 100% in FIG. 1. EP(3)0593 heterozygous flies show constantly a higher triglyceride content than the controls (100%; column 2 in FIG. 1. Therefore, the loss of gene activity in the locus 91D4 on chromosome 3R where the EP-vector of EP(3)0593 flies is heterozygous lethal integrated, is responsible for changes in the metabolism of the energy storage triglycerides, therefore representing an model for obese flies.

### Example 2

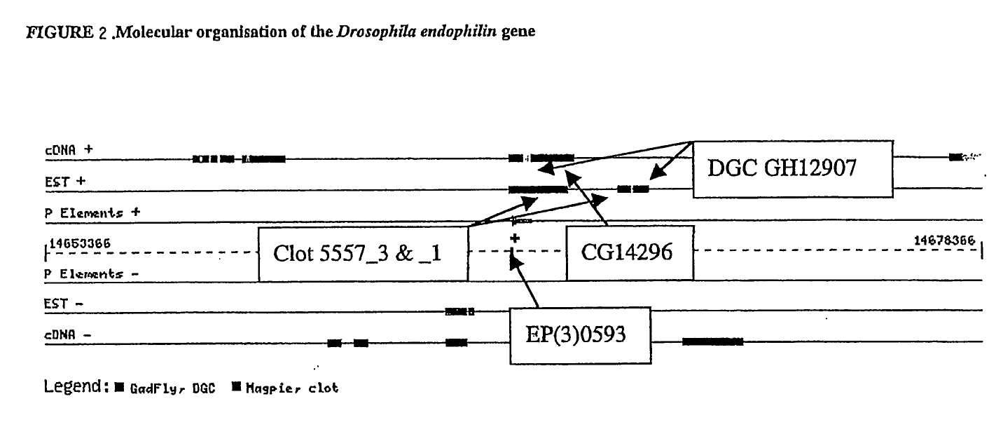

### Identification of a Drosophila Gene Responsible for the Change in Triglyceride Content (FIG. 2)

In FIG. 2, genomic DNA sequence is represented by the assembly as a dotted black line (from position 14653366 to 14678366 on chromosome 3R) that includes the integration sites of vector for lines EP(3)0593. Transcribed DNA sequences (ESTs) and predicted exons are shown as bars in the lower two lines. Predicted exons of the cDNA with GadFly Accession Number CG14296 are shown as dark grey bars and introns as light grey bars. Endophilin A encodes for a gene that is predicted by GadFly sequence analysis programs as Accession Number CG14296. Public DNA sequence databases (for example, NCBI GenBank or GadFly database) were screened thereby identifying the integration sites of lines EP(3)0593, causing an increase of triglyceride content. EP(3)0593 is integrated 45 base pairs of in the 5′ exon of the cDNA with Accession Number CG 14296. Therefore, expression of the cDNA encoding Accession Number CG14296 could be effected by heterozygous integration of vectors of lines EP(3)0593, leading to increase of the energy storage triglycerides.

### Example 3

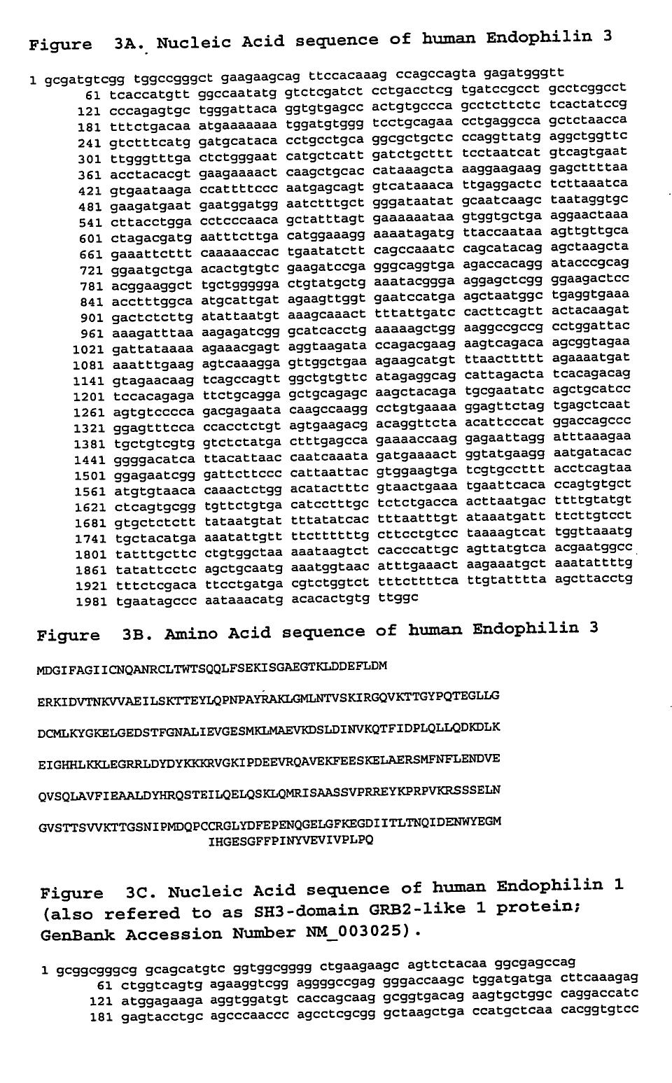

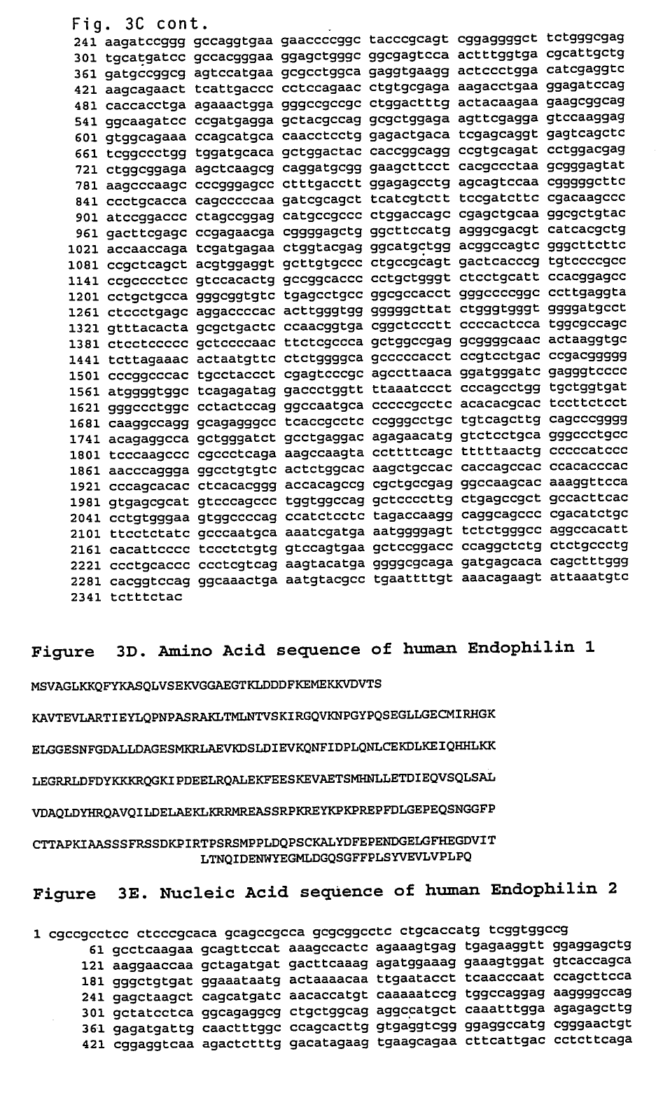

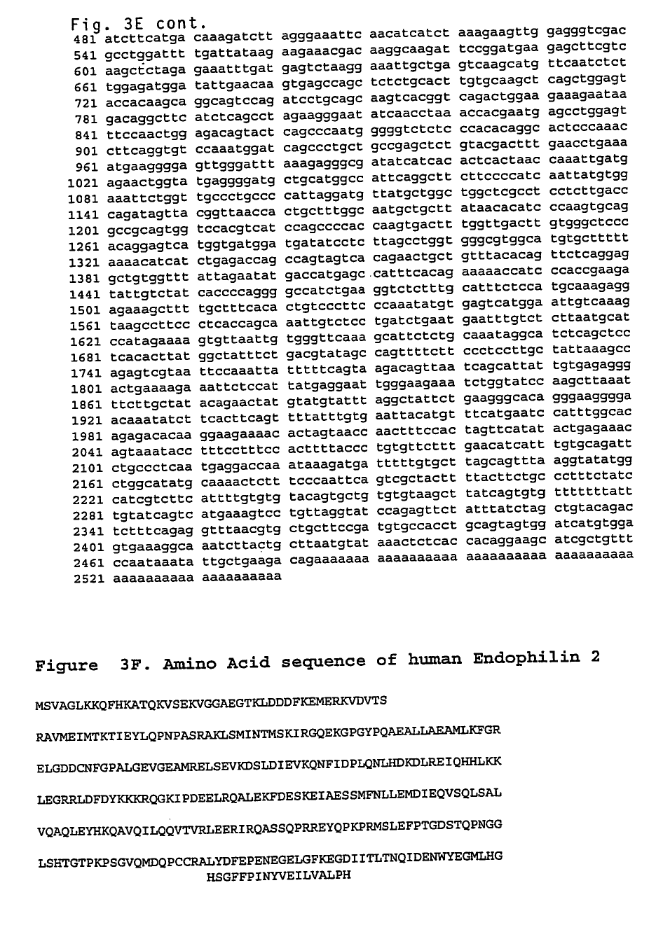

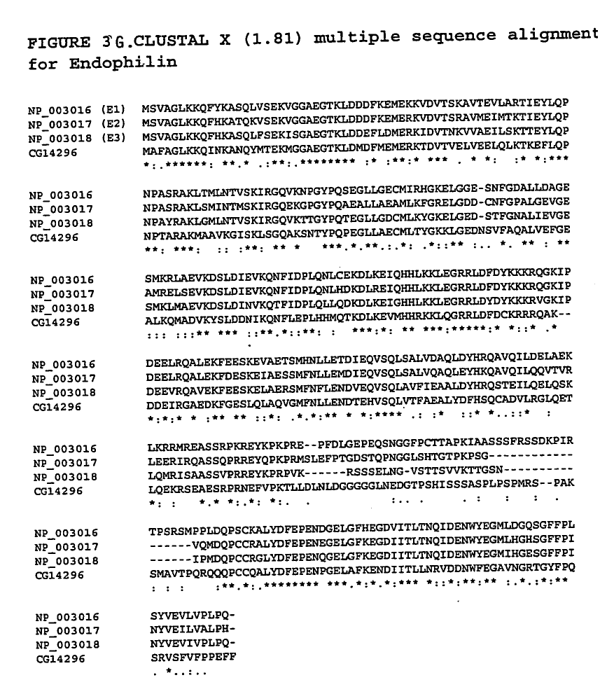

### Identification of Human Endophilin Homologues (FIG. 3)

Endophilin homologous proteins and nucleic acid molecules coding therefore are obtainable from insect or vertebrate species, e.g. mammals or birds. Particularly preferred are human Endophilin homologous nucleic acids, particularly nucleic acids encoding a human Endophilin 3 protein (Genbank Accession No. NM_{—}003027; Genbank Accession No. AF036269; EEN-B2-L1, SH3-domain GRB2-like 3), a human Endophilin 1 protein (Genbank Accession No. NM_{—}003025; Genbank Accession No. AF036268; EEN-B1, SH3-domain GRB2-like 1), or a human Endophilin 2 protein (Genbank Accession No. NM_{—}003026; Genbank Accession No. U65999; EEN, SH3-domain GRB2-like 2). Also preferred are mouse Endophilin homologous nucleic acids and polypeptides encoded thereby (Endophilin 3, GenBank Accession Number U58887; Endophilin 1, GenBank Accession Number U58886; or Endophilin 2, GenBank Accession Number U58885). Comparisons (Clustal X 1.8 analysis or Clustal W 1.82 analysis, see for example Thompson J. D. et al., (1 994) Nucleic Acids Res. 22(22):4673-4680; Thompson J. D., (1997) Nucleic Acids Res 25(24):4876-4882; Higgins, D. G. et al., (1996) Methods Enzymol. 266:383-402) between the respective proteins of different species (human, mouse, and Drosophila) were conducted and an alignment is shown in FIG. 3G.

### Example 4

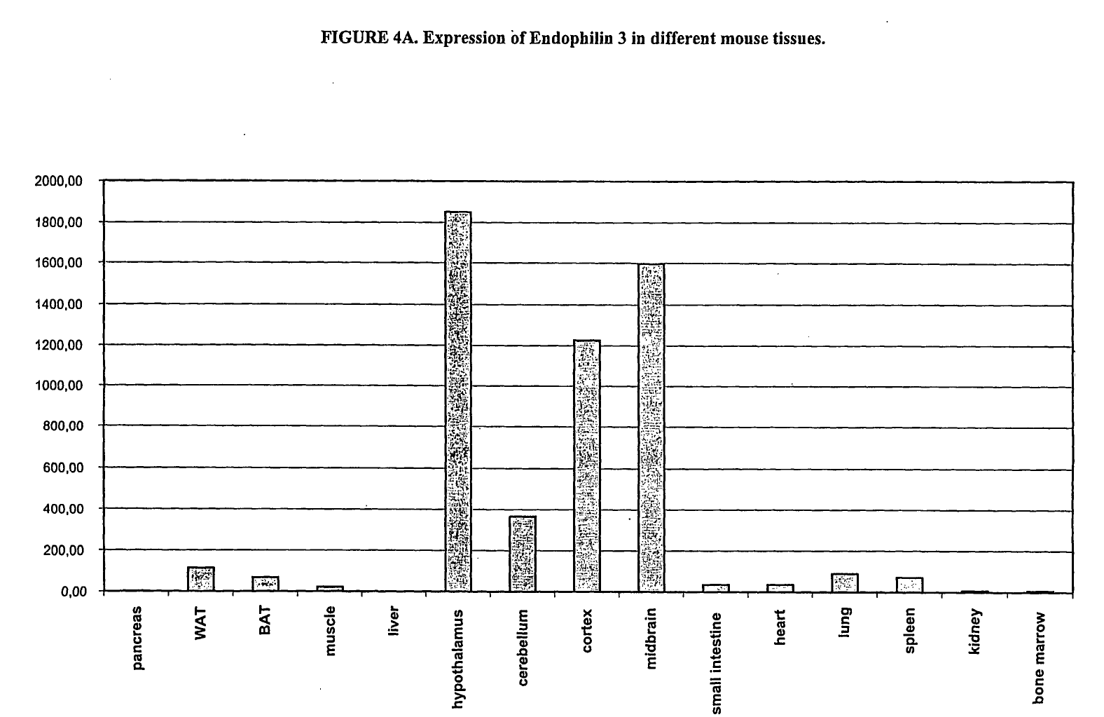

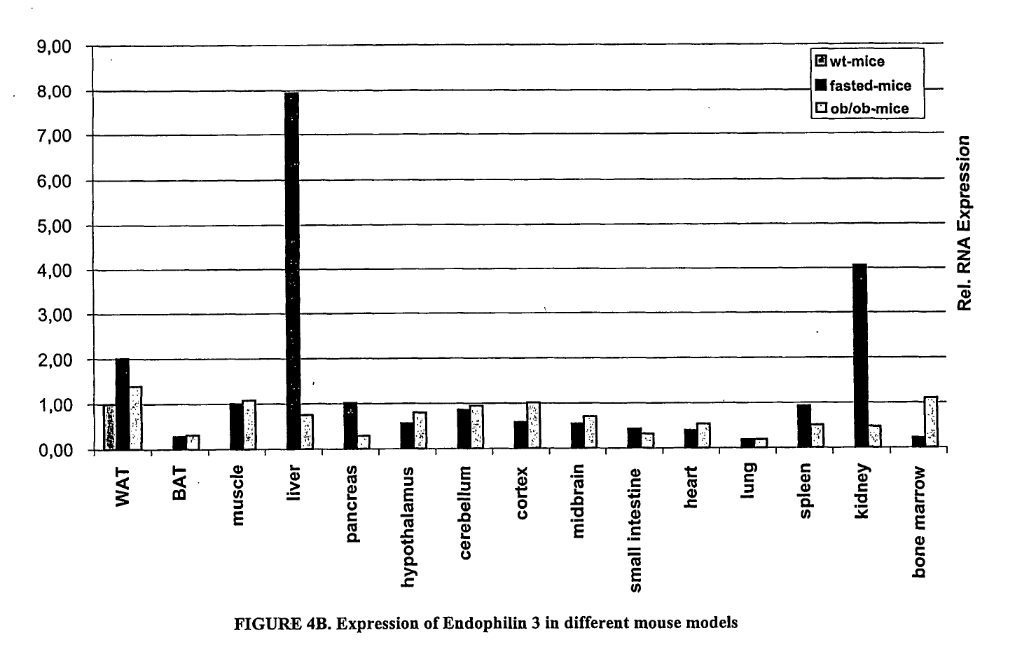

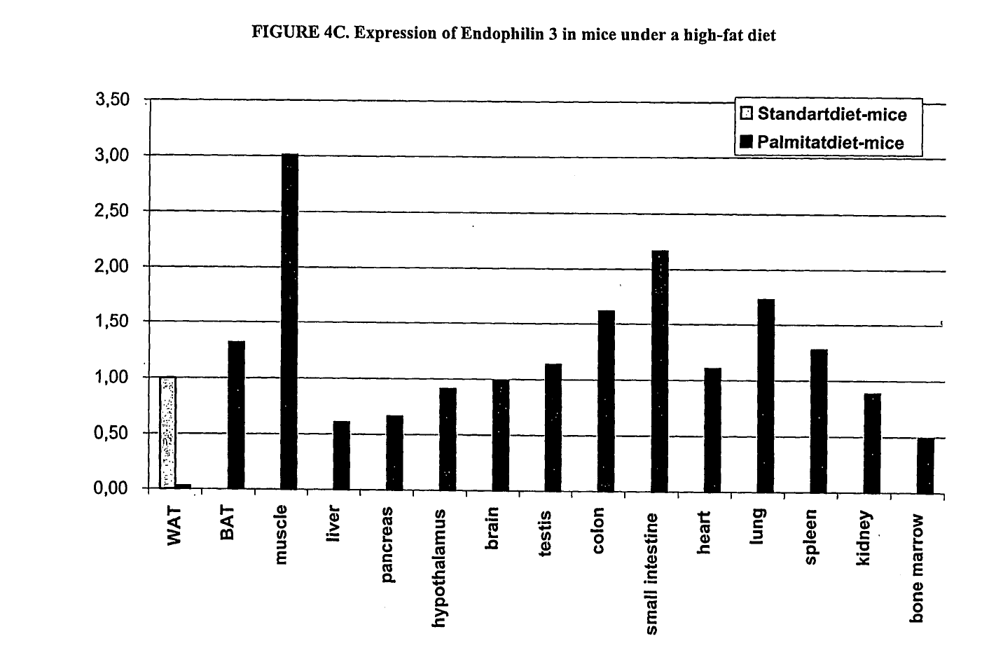

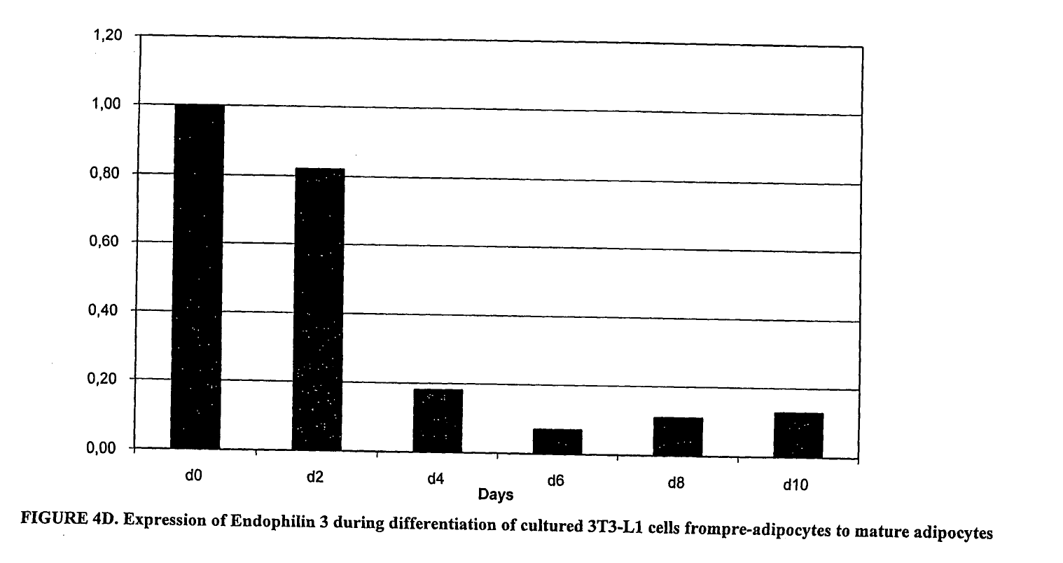

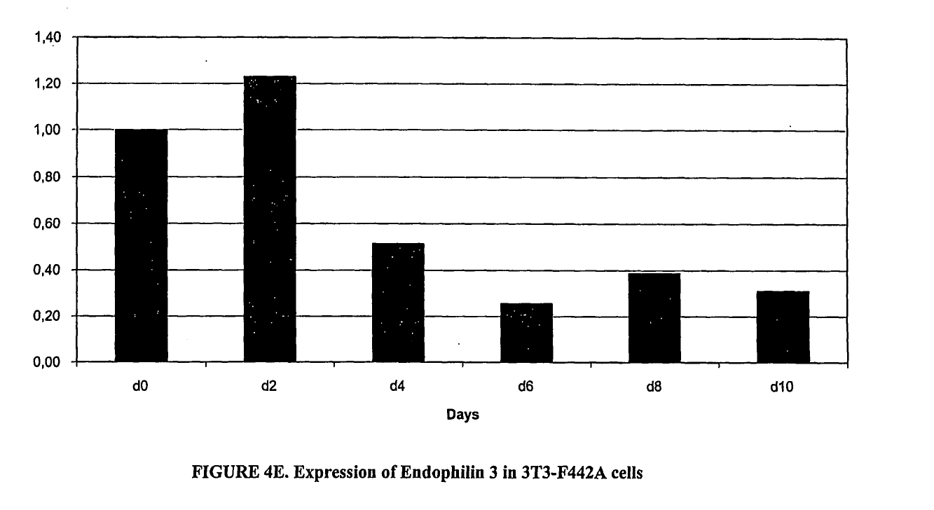

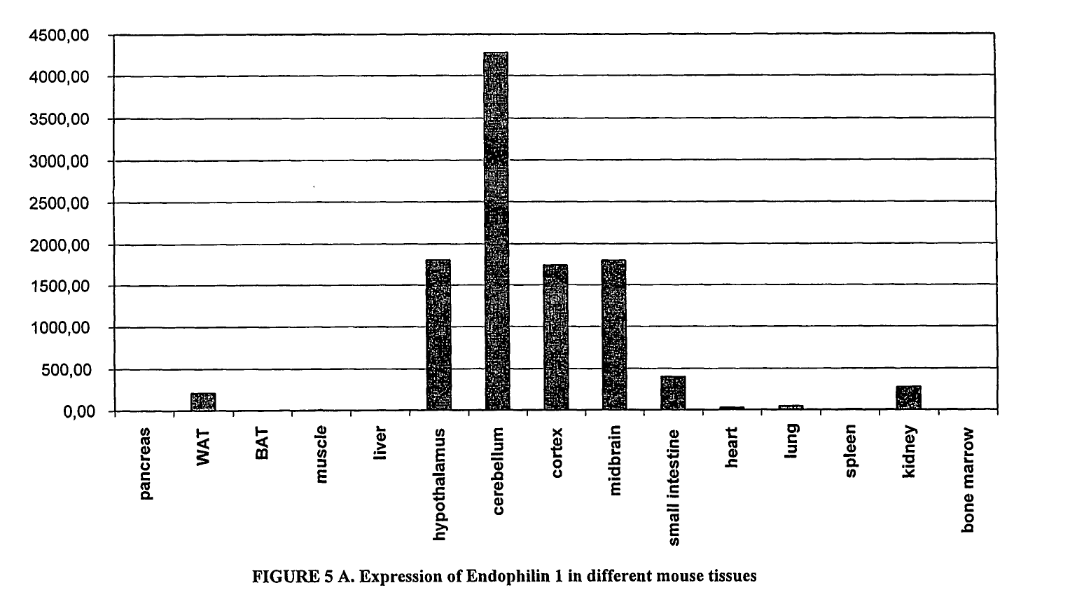

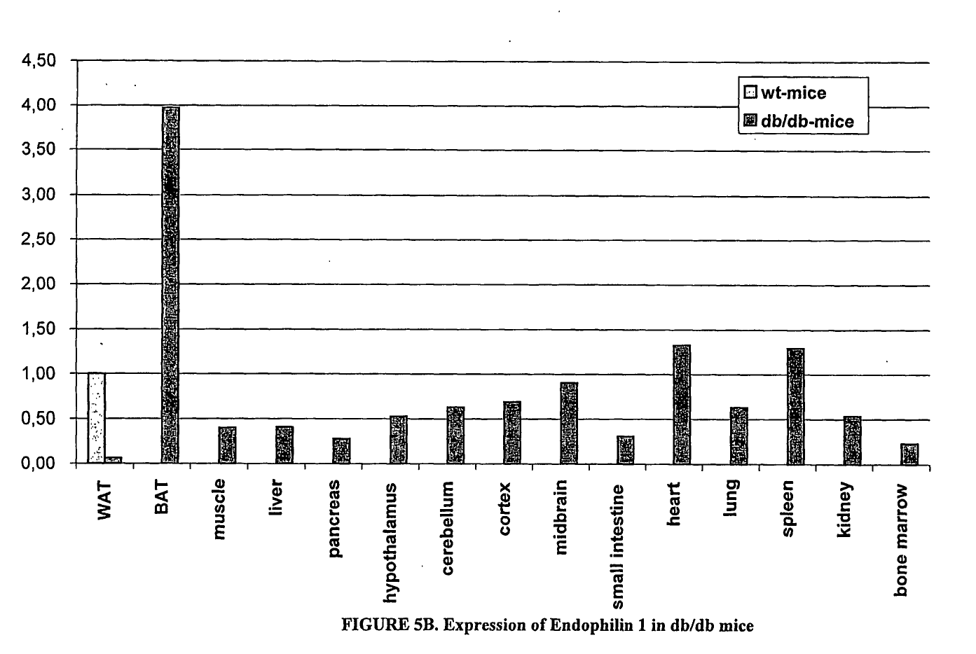

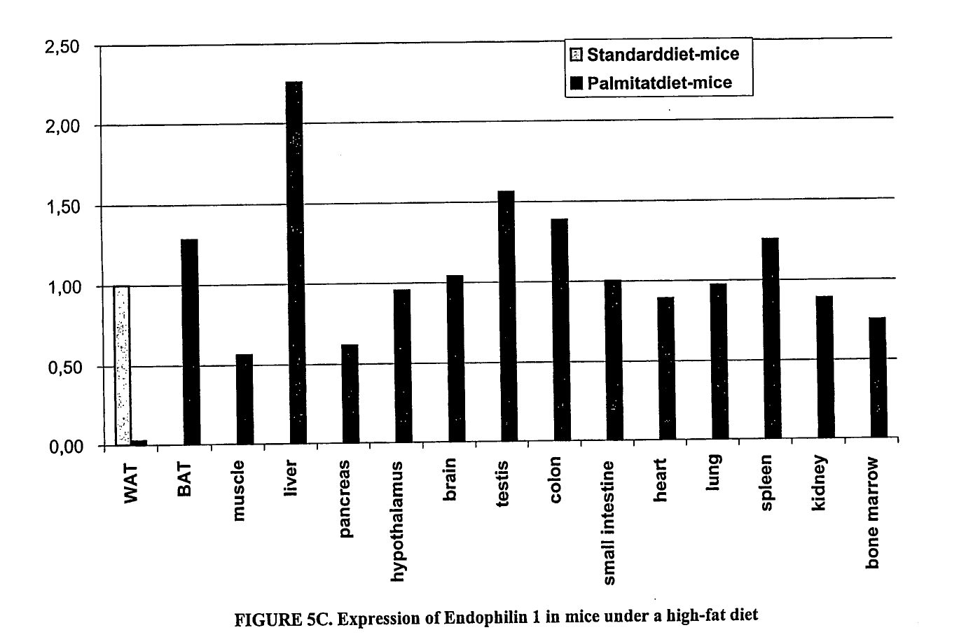

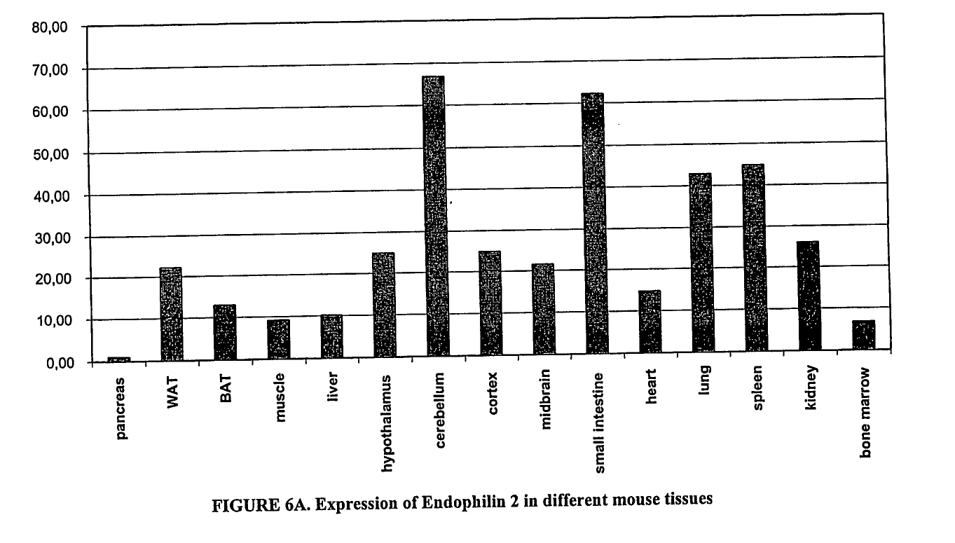

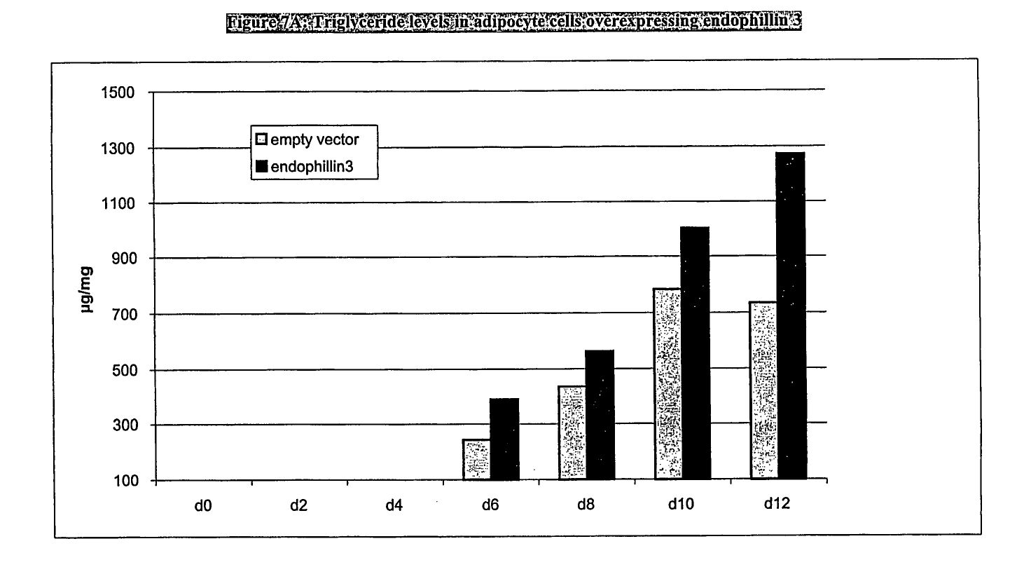

### Expression of Endophilin Polypeptides in Mammalian (Mouse) Tissues (FIG. 4, FIG. 5, and FIG. 6)

For analyzing the expression of Endophilins in mammalian tissues, several mouse strains (preferably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferably 22° C.), 40 per cent humidity and a light/dark cycle of preferably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum. (see, for example, Schnetzler et al. J Clin Invest July 1993; 92(1):272-80, Mizuno et al. Proc Natl Acad Sci USA Apr. 1996 16;93(8):3434-8). Animals were sacrificed at an age of 6 to 8 weeks. The animal tissues were isolated according to standard procedures known to those skilled in the art, snap frozen in liquid nitrogen and stored at −80° C. until needed.

For analyzing the role of Endophilins in the in vitro differentiation of different mammalian cell culture cells for the conversion of pre-adipocytes to adipocytes, mammalian fibroblast (3T3-L1) cells (e.g., Green & Kehinde, Cell 1: 1 13-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). In brief, cells were plated in DMEM/10% FCS (Invitrogen, Karlsruhe, Germany) at 50,000 cells/well in duplicates in 6-well plastic dishes and cultured in a humidified atmosphere of 5% CO2 at 37° C. At confluence (defined as day 0: d0) cells were transferred to serum-free (SF) medium, containing DMEM/HamF12 (3:1; Invitrogen), Fetuin (300 μg/ml; Sigma, Munich, Germany), Transferrin (2 μg/ml; Sigma), Pantothenate (17 μM; Sigma), Biotin (1 μM; Sigma), and EGF (0.8 nM; Hoffmann-La Roche, Basel, Switzerland). Differentiation was induced by adding Dexamethasone (DEX; 1 μM; Sigma), 3-Methyl-Isobutyl-1-Methylxanthine (MIX; 0.5 mM; Sigma), and bovine Insulin (5 μg/ml; Invitrogen). Four days after confluence (d4), cells were kept in SF medium, containing bovine Insulin (5 μg/ml) until differentiation was completed. At various time points of the differentiation procedure, beginning with day 0 (day of confluence) and day 2 (hormone addition; for example, dexamethason and 3-isobutyl-1-methylxanthin), up to 10 days of differentiation, suitable aliquots of cells were taken every two days.

Alternatively, mammalian fibroblast 3T3-F442A cells (e.g., Green & Kehinde, Cell 7: 105-113, 1976) were obtained from the Harvard Medical School, Department of Cell Biology (Boston, Mass., USA). 3T3-F442A cells were maintained as fibroblasts and differentiated into adipocytes as described previously (Djian, P. et al., J. Cell. Physiol., 124:554-556, 1985). At various time points of the differentiation procedure, beginning with day 0 (day of confluence and hormone addition, for example, Insulin), up to 1 0 days of differentiation, suitable aliquots of cells were taken every two days. 3T3-F442A cells are differentiating in vitro already in the confluent stage after hormone (insulin) addition.

For TaqMan Analysis of the proteins of the invention (FIG. 4, FIG. 5, and FIG. 6), RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferably using Superscript II RNaseH-Reverse Transcriptase, from Invitrogen, Karlsruhe, Germany) and subjected to Taqman analysis preferably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

For the analysis of the expression of Endophilin 3, Endophilin 1, and Endophilin 2, taqman analysis was performed using the following primer/probe pairs:

| | |
|---|---|
| Mouse Endophilin 3 forward primer | |
| 5′-AAC GAG TAA ATT GCG CCC AT-3′; | (Seq ID NO: 7) |
| Mouse Endophilin 3 reverse primer | |
| 5′-CAA AGG GTG CGT TCC CAC T-3′; | (Seq ID NO: 8) |
| Mouse Endophilin 3 Taqman probe | |
| (5/6-FAM) CGA ATG GCC TGG GTA GTC | (Seq ID NO: 9) |
| CTT GAC TG (5/6-TAMRA) | |
| Mouse Endophilin 1 forward primer | |
| 5′-TGC TTT GGT AAT GCT GCT TCC-3′; | (Seq ID NO: 10) |
| Mouse Endophilin 1 reverse primer | |
| 5′-GTG GGC TTG GTG ACT CAT CC-3′; | (Seq ID NO: 11) |
| Mouse Endophilin 1 Taqman probe | |
| (5/6-FAM) ACA TCA CGA ATG CAG GCC | (Seq ID NO: 12) |
| GCA G (5/6-TAMRA) | |
| Mouse Endophilin 2 forward primer | |
| 5′-CGA CGA GAA CTG GTA TGA GGG-3′; | (Seq ID NO: 13) |
| Mouse Endophilin 2 reverse primer | |
| 5′-GCA CGT AGC TGA GTG GGA AGA-3′; | (Seq ID NO: 14) |
| Mouse Endophilin 2 Taqman probe | |
| (5/6-FAM) ATG CTG CAC GGC CAA TCA | (Seq ID NO: 15) |
| GGC (5/6-TAMRA) | |

Expression profiling studies showed that Endophilin 3 and Endophilin 1 are clearly involved in the regulation of energy metabolism in mamals. In comparison to Endophilin 2, which is rather ubiquitously expressed, both Endophilin 3 and Endophilin 1 are more restricted in neuronal tissues. However, both proteins are also clearly expressed in WAT and BAT (FIG. 4A and FIG. 5A, respectively).

The expression of Endophilin 3 is strongly upregulated in liver of fasted mice (see FIG. 4B). In addition, a marked downregulation can be observed in brown adipose tissue of genetically obese (ob/ob) as well as of fasted mice (FIG. 4B). Endophilin 1 is strongly downregulated in white adipose tissue of genetically obese db/db mice (FIG. 5B).

The most prominent response with regard to metabolically active tissues can be seen in mice hold under a high fat diet: For Endophilin 3 as well as Endophilin 1, a dramatic decrease in their relative expression in white adipose tissue (WAT) can be observed in these mice (see FIG. 4C and FIG. 5C, respectively).

With regard to changes in expression intensity during the differentiation of preadipocytes to adipocytes, a strong reduction in relative signal intensity can be observed for Endophilin 3 during the in vitro differentiation program of 3T3-L1 as well as 3T3-F442A cells (see FIG. 4D and FIG. 4E).

### Example 5

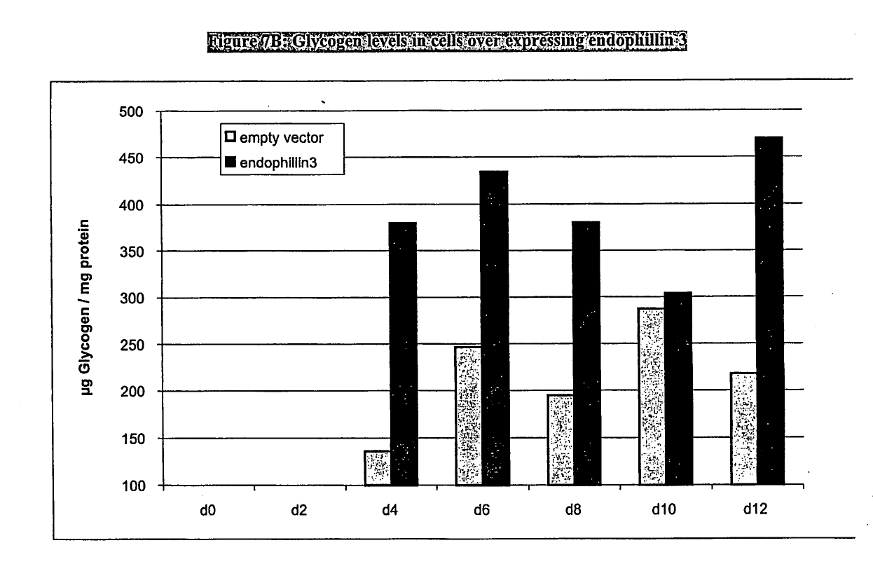

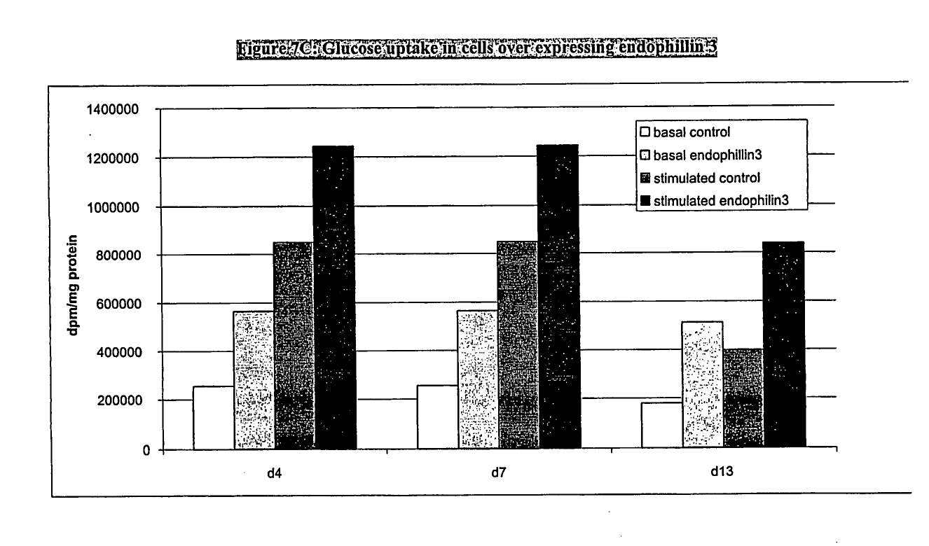

### Assays for the Determination of Triglyceride Storage, Synthesis and Transport (FIG. 7A)

Retroviral Infection of Preadipocytes

Packaging cells were transfected with retroviral plasmids pLPCX carrying mouse Endophillin transgene and a selection marker using calcium phosphate procedure. Control cells were infected with pLPCX carrying no transgene. Briefly, exponentially growing packaging cells were seeded at a density of 350,000 cells per 6-well in 2 ml DMEM+10% FCS one day before transfection. 10 min before transfection chloroquine was added directly to the overlying medium (25 μM end concentration). A 250 μl transfection mix consisting of 5 μg plasmid-DNA (candidate:helper-virus in a 1:1 ratio) and 250 mM CaCl₂ was prepared in a 15 ml plastic tube. The same volume of 2×HBS (280 μM NaCl, 50 μM HEPES, 1.5 mM Na₂HPO₄, pH 7.06) was added and air bubbles were injected into the mixture for 15 sec. The transfection mix was added drop wise to the packaging cells, distributed and the cells were incubated at 37° C., 5% CO₂ for 6 hours. The cells were washed with PBS and the medium was exchanged with 2 ml DMEM+10% CS per 6-well. One day after transfection the cells were washed again and incubated for 2 days of virus collection in 1 ml DMEM+10% CS per 6-well at 32° C., 5% CO₂.

The supernatant was then filtered through a 0.45 μm cellulose acetate filter and polybrene (end concentration 8 μg/ml) was added. Mammalian fibroblast (3T3-L1) cells in a sub-confluent state were overlaid with the prepared virus containing medium. The infected cells were selected for 1 week with 2 μg/ml puromycin. Following selection the cells were checked for transgene expression by western blot and immunofluorescence. Over expressing cells were seeded for differentiation.

3T3-L1 cells were maintained as fibroblasts and differentiated into adipocytes as described in the prior art and in example 8. For analysing the role of the proteins disclosed in this invention in the in vitro assays for the determination of triglyceride storage, synthesis and transport were performed.

Preparation of Cell Lysates for Analysis of Metabolites

Starting at confluence (D0), cell media was changed every 48 hours. Cells and media were harvested 8 hours prior to media change as follows. Media was collected, and cells were washed twice in PBS prior to lyses in 600 microl HB-buffer (0.5% Polyoxyethylene 10 tridecylethane, 1 mM EDTA, 0.01M NaH2PO4, pH 7.4). After inactivation at 70° C. for 5 minutes, cell lysates were prepared on Bio 101 systems lysing matrix B (0.1 mm silica beads; Q-Biogene, Carlsbad, USA) by agitation for 2×45 seconds at a speed of 4.5 (Fastprep FP120, Bio 101 Thermosavant, Holbrock, USA). Supernatants of lysed cells were collected after centrifugation at 3000 rpm for 2 minutes, and stored in aliquots for later analysis at −80° C.

Changes in cellular triglyceride levels during adipogenesis (FIG. 7A) Cell lysates and media were simultaneously analysed in 96-well plates for total protein and triglyceride content using the Bio-Rad DC Protein assay reagent (Bio-Rad, Munich, Germany) according to the manufacturer's instructions and a modified enzymatic triglyceride kit (GPO-Trinder; Sigma) briefly final volumes of reagents were adjusted to the 96-well format as follows: 10 μl sample was incubated with 200 μl reagent A for 5 minutes at 37° C. After determination of glycerol (initial absorbance at 540 nm), 50 μl reagent B was added followed by another incubation for 5 minutes at 37° C. (final absorbance at 540 nm). Glycerol and triglyceride concentrations were calculated using a glycerol standard set (Sigma) for the standard curve included in each-assay.

As shown in FIG. 7A, we found that in Endophilin 3 over-expressing cells s cellular triglyceride levels were increased throughout adipogenesis.

### Example 6

### Changes in Cellular Glycogen Levels During Adipogenesis (FIG. 7B)

Cell lysates and media were simultaneously analysed in triplicates in 96-well plates for total protein and glycogen content using the Bio-Rad DC Protein assay reagent (Bio-Rad, Munich, Germany) according to the manufacturer's instructions and an enzymatic starch kit from Hoffmann-La Roche (Basel, Switzerland). 10-μl samples were incubated with 20-μl amyloglucosidase solution for 15 minutes at 60° C. to digest glycogen to glucose. The glucose is further metabolised with 100μl distilled water and 100 μl of enzyme cofactor buffer and 12 μl of enzyme buffer (hexokinase and glucose phosphate dehydrogenase). Background glucose levels are determined by subtracting values from a duplicate plate without the amyloglucosidase. Final absorbance is determined at 340 nm. HB-buffer as blank, and a standard curve of glycogen (Hoffmann-La Roche) were included in each assay. Glycogen content in samples was calculated using a standard curve.

As shown in FIG. 7B, we found that in Endophilin 3 over-expressing cells cellular glycogen levels were increased throughout adipogenesis. Glycogen levels in cells are more variable than triglyceride levels because the turnover of glycogen is higher. Glucose is taken up by the cells rapidly and stored in the form of glycogen. This energy storage is than primarily used for the metabolic demands of the cell. The increase in glycogen levels when over-expressing Endophilin 3 could be the effect of a decreased metabolic rate in these cells or an increase in glucose uptake as compared to empty vector transduced cells.

### Example 7

### Glucose Uptake Assay (FIG. 7C)

For the determination of glucose uptake, cells were washed 3 times with PBS prior to serum starvation in Krebs-Ringer-Bicarbonate-Hepes buffer (KRBH; 134 nM NaCl, 3.5 mM KCl, 1.2 mM KH₂PO₄, 0.5 mM MgSO₄, 1.5 mM CaCl₂, 5 mM NaHCO₃, 10 mM Hepes, pH 7.4), supplemented with 0.1% FCS and 0.5 mM Glucose for 2.5 h at 37° C. For insulin-stimulated glucose uptake, cells were incubated with 1 μM bovine insulin (Sigma; carrier: 0.005 N HCl) for 45 min at 37° C. Basal glucose uptake was determined with carrier only. Non-metabolizable 2-Deoxy-3H-D-Glucose (NEN Life Science, Boston, USA) in a final activity of 0.4 μCi/Well/ml was added for 30 min at 37° C. For the calculation of background radioactivity, 25 μM Cytochalasin B (Sigma) was used. All assays were performed in duplicate wells. To terminate the reaction, cells were washed 3 times with ice cold PBS, and lysed in 1 ml 0.1N NaOH. Protein concentration of each well was assessed using the standard Biuret method (Protein assay reagent; Bio-Rad), and scintillation counting of cell lysates in 10 volumes Ultima-gold cocktail (Packard Bioscience, Groningen, Netherlands) was performed.

As shown in FIG. 7C the basal glucose uptake of adipocyte (for example, 3T3-L1) cells over-expressing Endophilin 3 is increased significantly by more than 100% during adipogenesis at all times. This effect is already visible in the pre-adipocyte (d4) and also in a fully differentiated adipocyte (d13). This increase in glucose and therefore energy uptake of the cells is most likely the reason for the increased glycogen and triglyceride levels during adipocyte (e.g., 3T3-L1) differentiation (see FIGS. 7a and 7b). Endophilin 3 does not seem to influence insulin stimulated glucose uptake but clearly has an effect on the glucose uptake of adipocytes, confirming its role in diabetes and related metabolic disorders.

### Example 8

### Generation and Analysis of Endophilin 3 Transgenic Animals (aP2-Endophilin 3)

Generation of the Transgenic Animals

Mouse Endophilin 3 cDNA was isolated from mouse midbrain tissue using standard protocols as known to those skilled in the art. The cDNA was amplified by RT-PCR using the following primer pair:

| | |
|---|---|
| mEndophilin 3 forward primer: | |
| 5′ GGC GCC GCC GCG ATG TCG GTG 3′ | (SEQ ID NO: 16) |
| mEndophilin 3 reverse primer: | |
| 5′ TAG GTC CAA AAG ACA CAT TTA CGG | (SEQ ID NO: 17) |
| AGG 3′. | |

The resulting mEndophilin 3 cDNA was cloned into the Smal site of pTG-aP2-X-hgh-bgh-polyA (Develogen AG), a derivative of pBluescript KS+ (Stratagene) containing additional restriction sites in the polylinker, an aP2-promoter/enhancer fragment (Graves et al., 1991), an exon/intron structure derived from the human growth hormone gene and a bovine polyadenylation signal according to standard protocols, resulting in a plasmid referred to as pTG-aP2-mEndophilinlll-hgh-bgh-polyA′.

The aP2-Endophilin 3 transgene was microinjected into the male pronucleus of fertilized mouse embryos (preferable strain FVB (Harlan Winkelmann)). Injected embryos were transferred into pseudo-pregnant foster mice. Transgenic founders were detected by PCR analysis using an aP2 forward primer (SEQ ID NO: 18): 5′ TGC CAG GGA GAA CCA AAG T 3′ and Endophilin 3 reverse primer (SEQ ID NO: 19): 5′ GCC TCA CCA ACA TCT ACC AAC 3′. Transgenic mouse lines containing the aP2-Endophilin 3 construct were established. Briefly, founder animals were backcrossed with C57/BL6 mice to generate F1 mice for analysis. Transgenic mice were continously bred onto the C57/B16 background.

Analysis of the Bodyweight of the Transgenic Mice

After weaning, male aP2-Endophilin transgenic mice and their wild-type (wt) littermates controls were placed in groups of 4 to 5 animals (N=4 up to N=5) on control diet (preferably Altromin C1057 mod control! 4.5% crude fat) or high fat diet (preferably Altromin C1057 mod. high fat, 23.5% crude fat). Total body weight of the animals was measured weekly over a period of 12-16 weeks.

1. A pharmaceutical composition comprising a nucleic acid molecule of the Endophilin gene family or a polypeptide encoded thereby or a fragment or a variant of said nucleic acid molecule or said polypeptide or an antibody, an aptamer or another receptor recognizing a nucleic acid molecule of the Endophilin gene family or a polypeptide encoded thereby together with pharmaceutically acceptable carriers, diluents and/or adjuvants.

2. The composition of claim 1, wherein the nucleic acid molecule is a vertebrate or insect Endophilin nucleic acid, particularly a human Endophilin homologous nucleic acids, particularly nucleic acids encoding a human Endophilin 3 protein (Genbank Accession No. NM_{—}003027; Genbank Accession No. AF036269), a human Endophilin 1 protein (Genbank Accession No. NM_{—}003025; Genbank Accession No. AF036268) or a human Endophilin 2 protein (Genbank Accession No. NM_{—}003026; Genbank Accession No. U65999) and mouse Endophilin homologous nucleic acids and polypeptides encoded thereby (Endophilin 3, GenBank Accession Number U58885; Endophilin 1, GenBank Accession Number U58887; or Endophilin 2, GenBank Accession Number U58885;), or a fragment thereof or a variant thereof.

3. The composition of claim 1 wherein said nucleic acid molecule
(a) hybridizes at 50° C. in a solution containing 1×SSC and 0.1% SDS to a nucleic acid molecule encoding as specified in claim 2 or a nucleic acid molecule which is complementary thereto;
(b) it is degenerate with respect to the nucleic acid molecule of (a);
(c) encodes a polypeptide which is at least 85%, preferably at least 90%, more preferably at least 95%, more preferably at least 98% and up to 99.6% identical to an Endophilin as defined in claim 2;
(d) differs from the nucleic acid molecule of (a) to (c) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of an disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and others, in cells, cell masses, organs and/or subjects.

14. The composition of claim 13 for the manufacture of an agent for modulating metabolic processes, particularly metabolite transport in adipocytes or adipogenesis.

15. Use of a nucleic acid molecule of the Endophilin gene family or a polypeptide encoded thereby or a fragment or a variant of said nucleic acid molecule or said polypeptide or an antibody, an aptamer or another receptor recognizing a nucleic acid molecule of the Endophilin gene family or a polypeptide encoded thereby for controlling the function of a gene and/or a gene product which is influenced and/or modified by an Endophilin homologous polypeptide.

16. Use of the nucleic acid molecule of the Endophilin gene family or a polypeptide encoded thereby or a fragment or a variant of said nucleic acid molecule or said polypeptide or an antibody, an aptamer or another receptor recognizing a nucleic acid molecule of the Endophilin gene family or a polypeptide encoded thereby for identifying substances capable of interacting with an Endophilin homologous polypeptide.

17. A non-human transgenic animal exhibiting a modified expression of an Endophilin polypeptide.

18. The animal of claim 17, wherein the expression of the Endophilin polypeptide is increased and/or reduced.

19. A recombinant host cell exhibiting a modified expression of an Endophilin polypeptide.

20. The cell of claim 19 which is a human cell.

21. A method of identifying a (poly)peptide involved in the regulation of energy homeostasis and/or metabolism of triglycerides in a mammal comprising the steps of
(a) contacting a collection of (poly)peptides with an Endophilin polypeptide or a fragment thereof under conditions that allow binding of said (poly)peptides;
(b) removing (poly)peptides which do not bind and
(c) identifying (poly)peptides that bind to said Endophilin polypeptide or fragment thereof.

22. The method of claim 21, wherein the (poly)peptides bind to said Endophilin polypeptide or fragment thereof via an SH3 domain-mediated interaction.

23. A method of screening for an agent which modulates the interaction of an Endophilin polypeptide with a binding target/agent, comprising the steps of
(a) incubating a mixture comprising
(aa) an Endophilin polypeptide, or a fragment thereof;
(ab) a binding target/agent of said Endophilin homologous polypeptide or fragment thereof; and
(ac) a candidate agent
under conditions whereby said Endophilin polypeptide or fragment thereof specifically binds to said binding target/agent at a reference affinity;
(b) detecting the binding affinity of said Endophilin polypeptide or fragment thereof to said binding target to determine a (candidate) agent-biased affinity; and
(c) determining a difference between (candidate) agent-biased affinity and reference affinity.

24. The method of claim 23 wherein the binding target/agent binds to the Endophilin polypeptide or fragment thereof via an SH3 domain-mediated interaction.

25. The method of claim 23 wherein the agent is a small molecular modulator, a nucleic acid modulator or an antibody.

26. A method of screening for an agent which modulates the activity of an Endophilin polypeptide, comprising the steps of
(a) incubating a mixture comprising
(aa) an Endophilin polypeptide, or a fragment thereof and
(ab) a candidate agent
under conditions whereby said Endophilin polypeptide or fragment thereof exhibits a reference activity;
(b) detecting the activity of said Endophilin polypeptide or a fragment thereof to determine a (candidate) agent-biased activity and
(c) determining a difference between (candidate) agent-biased activity and reference activity.

27. The method of claim 26, wherein the agent is a small molecular modulator, a nucleic acid modulator or an antibody.

28. (canceled)

29. (canceled)

30. Use of a (poly)peptide as identified by the method of claim 21 for the preparation of a pharmaceutical composition for the treatment, alleviation and/or prevention of of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.

31. Use of a nucleic acid molecule of the Endophilin gene family or of a fragment thereof for the preparation of a non-human animal which over- or under-expresses the Endophilin gene product.

32. Kit comprising at least one of
(a) an Endophilin nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

33. Use of an agent as identified by the method of claim 23 for the preparation of a pharmaceutical composition for the treatment, alleviation and/or prevention of of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.
